(19) United States
(12) Patent Application Publication
(10) Pub. No.: US 2011/0082704 A1
(43) Pub. Date: Apr. 7, 2011

(54) Systems, Devices, and/or Methods for Managing Healthcare Information

(76) Inventor: Ronald D. Blum, Roanoke, VA (US)

(21) Appl. No.: 12/887,591

(22) Filed: Sep. 22, 2010

Related U.S. Application Data
(60) Provisional application No. 61/248,980, filed on Oct. 6, 2009.

(51) Int. Cl. G06Q 50/00; A61B 3/032

(52) U.S. Cl. 705/2; 351/239

(57) ABSTRACT

Certain exemplary embodiments can provide a system, machine, device, manufacture, circuit, composition of matter, and/or user interface adapted for and/or resulting from, and/or a method and/or machine-readable medium storing machine-implementable instructions for, activities that can comprise and/or relate to, receiving, by an intermediary from a product provider: information regarding a product, and/or compensation for providing, to the product provider, information regarding a recommendation of the product.

### CROSS-REFERENCES TO RELATED APPLICATIONS

This application claims priority to, and incorporates by reference herein in its entirety, pending U.S. Provisional Patent Application 61/248,980, filed 6 Oct. 2009.

### BRIEF DESCRIPTION OF THE DRAWINGS

A wide variety of potential practical and useful embodiments will be more readily understood through the following detailed description of certain exemplary embodiments, with reference to the accompanying exemplary drawings in which:

FIG. 1 is a block diagram of an exemplary embodiment of a system;

FIG. 2 is a block diagram of an exemplary embodiment of a system;

FIG. 3 is a screen shot of an exemplary embodiment of a user interface;

FIG. 4 is a screen shot of an exemplary embodiment of a user interface;

FIG. 5 is a screen shot of an exemplary embodiment of a user interface;

FIG. 6 is a screen shot of an exemplary embodiment of a user interface;

FIG. 7 is a screen shot of an exemplary embodiment of a user interface;

FIG. 8 is a screen shot of an exemplary embodiment of a user interface;

FIG. 9 is a screen shot of an exemplary embodiment of a user interface;

FIG. 10 is a screen shot of an exemplary embodiment of a user interface;

FIG. 11 is a screen shot of an exemplary embodiment of a user interface;

FIG. 12 is a screen shot of an exemplary embodiment of a user interface;

FIG. 13 is a screen shot of an exemplary embodiment of a user interface;

FIG. 14 is a screen shot of an exemplary embodiment of a user interface;

FIG. 15 is a screen shot of an exemplary embodiment of a user interface;

FIG. 16 is a screen shot of an exemplary embodiment of a user interface;

FIG. 17 is a screen shot of an exemplary embodiment of a user interface;

FIG. 18 is a screen shot of an exemplary embodiment of a user interface;

FIG. 19 is a screen shot of an exemplary embodiment of a user interface;

FIG. 20 is a screen shot of an exemplary embodiment of a user interface;

FIG. 21 is a screen shot of an exemplary embodiment of a user interface;

FIG. 22 is a screen shot of an exemplary embodiment of a user interface;

FIG. 23 is a screen shot of an exemplary embodiment of a user interface;

FIG. 24 is a screen shot of an exemplary embodiment of a user interface;

FIG. 25 is a screen shot of an exemplary embodiment of a user interface;

FIG. 26 is a screen shot of an exemplary embodiment of a user interface;

FIG. 27 is a screen shot of an exemplary embodiment of a user interface;

FIG. 28 is a screen shot of an exemplary embodiment of a user interface;

FIG. 29 is a screen shot of an exemplary embodiment of a user interface;

FIG. 30 is a screen shot of an exemplary embodiment of a user interface;

FIG. 31 is a flowchart of an exemplary embodiment of a method;

FIG. 32 is a flowchart of an exemplary embodiment of a method;

FIG. 33 is a flowchart of an exemplary embodiment of a method;

FIG. 34 is a flowchart of an exemplary embodiment of a method;

FIG. 35 is a flowchart of an exemplary embodiment of a method;

FIG. 36 is a block diagram of an exemplary embodiment of a system;

FIG. 37 is a block diagram of an exemplary embodiment of an information device; and

FIG. 38 is a flowchart of an exemplary embodiment of a method.

### DETAILED DESCRIPTION

As the world ages and more and more people require eye examinations and glasses there is expected to be continued pressure to speed patients through the eye doctor's and eye care professional's offices. By this very nature chair time/doctor time spent with the patient is likely to continue to decrease. Unfortunately even if the quality of the eye examination does not suffer from this (and it is unclear if this is occurring or not), what is likely to suffer is the quality of guidance that the eye doctor or eye care professional is providing to the patient.

Eye doctors (ophthalmologists and optometrists) sometimes dilate and/or cycloplegea patient's eyes to better detect eye disease. The drops used for the routine eye examination can retard and/or suppress the patient's ability to accommodate (focus) and/or see clearly at near distances. This can present a major challenge for eye doctors who prescribe eye glasses and sell them to patients at their offices. If the doctor begins the examination, but waits to dilate the eyes of a patients until after the patient picks out eyeglasses, the patient will be able to see clearly for the eyeglass selection but then the patient must be re-examined for eye disease before departing the office. This can be very distracting and disruptive to the patient flow of the practice. On the other hand, if the patient is dilated at the start of the eye examination or during the eye examination, then the patient likely will be unable to read written material presented to the patient at the end of the eye examination. Thus, it can be very difficult for the patient to pick out their eyeglass frames or to read material that is healthcare related or related to the doctor's recommendations after being dilated. Consequently, there can be a need to be able to efficiently communicate the doctor's recommendation to the patient in an effective manner even though the patient's vision is blurred due to the dilation and cycloplegic eye drops that are used for a routine eye examination.

In today's world the eye doctor or eye care professional upon finishing the eye examination often quickly writes a prescription for eye glasses, or contact lenses (whether by way of computer or hand), if needed and sends the patient with the prescription to the optical boutique for eye glasses or contact lenses. In certain cases the patient goes to an optical boutique/dispensary within the office of the eye doctor or eye care professional, and in other cases the patient takes the eye glass prescription to a non-affiliated 3rd party optical boutique/dispensary. But in all cases the amount of education and advice the patient receives from the eye doctor or eye care professional regarding the type of lens that is being prescribed is typically handed off downstream to that of the optician within the optical boutique/dispensary. This loss of education and advice is very unfortunate. While the eye doctor or eye care professional speeds to see his or her next patient the patient is now hostage to the handoff of his or her prescription and totally dependent upon the quality of care they will now receive at the optical boutique/dispensary.

In most cases it is believed by the inventor (who has been in the optical industry at all levels for the last 30 yrs) that the quality of care rendered the patient at the optical boutique/dispensary is quite good. But in too many cases the quality of advice is lacking. And as with the eye doctor/eye care professional office depending upon the time of day, the number of patients waiting, the loyalty of the optician who owns or works at the optical dispensary to that of certain lens manufacturers, spiffs paid to the optician by the lens manufacturers, the patient may or may not end up being told or advised about the best lens solution for the patient.

Certain exemplary embodiments can communicate the doctor's recommendation effectively and/or efficiently to the patient by way of allowing the information device to be attached to the near point rod of the phoropter/refractor. If the patient has been dilated and cyclopleged and their accommodative system fatigued or retarded by the eye drops, certain exemplary embodiments can allow the patient to see and/or hear the doctor's product recommendations clearly (which can be rendered via the information device) by way of adding additional plus lenses into the phoropter/refractor as the patient looks through these lenses. These additional lenses can off-set the fatiguing and/or retarding effect that the eye drops have on the patient's accommodative system.

In certain exemplary embodiments, the information device can be detachable from the near point rod of the phoropter and carried throughout the office.

In certain exemplary embodiments, the information device need not be attached to the near point rod of the phoropter.

Today there are numerous (hundreds) of lens solutions available to the patient which can greatly help with the quality of vision and in certain circumstances quality of life. For example, there are progressive addition lenses that can provide clear uninterrupted vision from far to near and back again but can have limitations of clarity of horizontal width and/or can have associated unwanted astigmatism and distortion. There are new dynamic lens choices such as electronic focusing lenses that can focus without movement. There are enhanced multifocals available that can allow for very clear wide vision in the distance, less optical magnification jump than that of a bifocal of equal power, and/or 10× the amount of clear vision within 5 feet of the patient/wearer, but due to the soft power jump will not necessarily allow for clear interrupted vision from far to near seamlessly and back. There are the standard bifocals and trifocals that have been sold for the last 100 years or so. There are numerous types of coatings and lens treatments available that can enhance the performance of the lenses such as: anti-reflection coatings; photo-chromatic coatings; hydro-phobic coatings; scratch resistance coatings; ultra-violet inhibition; and/or soon, selective blue light filtering. Each of these, or a combination, depending upon the patient's vision, occupation, health, and/or life-style, can be of great benefit to a specific patient.

Therefore there can be a pressing need for a system, device, and/or method that can allow for more consistency of care and/or proper advice while allowing the eye doctor or eye care provider to seamlessly incorporate such a system, device, and/or method into their practice, and/or the manner in which they care for their patients, without slowing down their patient flow and/or increasing the time the eye doctor or eye care provider has to spend with the patient.

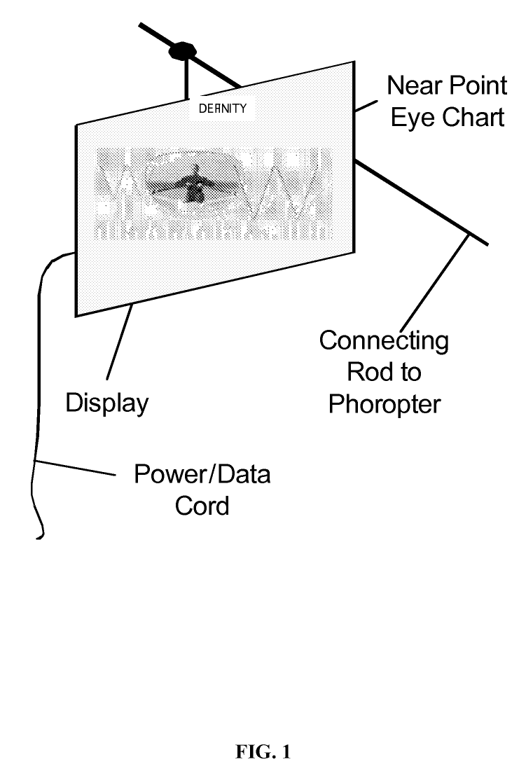

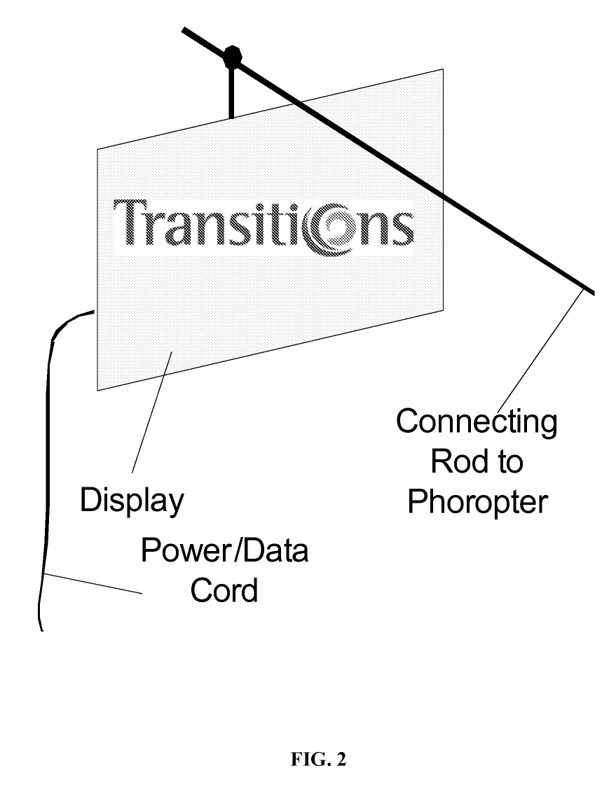

As shown in FIGS. 1 and 2, certain exemplary embodiments can provide an eye examination near point electronic display system that is affixed to a near point rod attached to the eye examination phoropter/refractor. The eye examination near point electronic display system can complement and/or replace a near point card.

Certain exemplary embodiments can include an audio means. When the audio means is provided, a speaker or speakers can be associated with the electronic display system remotely, or attached directly to, on, or in the electronic display. The electronic display can have thin, mostly flat, speakers affixed thereto.

The electronic display can be capable of displaying static or dynamic/moving color and/or mono-chromatic images. The images can take the form of still pictures and/or streaming video that is down-loaded (potentially along with audio) from, by way of example only, the web, internet, WIFI, local networks, and/or satellite, directly to the electronic display, and/or to a memory device located within, on, and/or near the electronic display and/or connected to the electronic display. The images and audio can be taken from a memory stick and/or card.

The electronic display can be made from any type of display format, such as by way of example only, LCD, OLED, plasma, electronic ink, electronic paper, etc. The size of the electronic display can be of any size and/or shape such that it is capable of being affixed to the near point rod of a phoropter/refractor. For example, the size of the display can be approximately 4 inches high by 6 inches in width, 5 inches wide by 7 inches high, etc. The display system can be located approximately 10 inches to approximately 36 inches (such as approximately 14 to approximately 20 inches) from the phoropter/refractor.

The electronic display and/or audio speaker or speakers can be powered by any source generally used to power video displays such as, by way of example only, batteries, and/or conventional AC/DC electrical power adaptors. In certain exemplary embodiments the electronic display and speakers can be connected by way of a power cord that is located on or within part of the near point rod and/or that extends from the electronic display on or thru the near point rod to the phoropter/refractor housing, across a portion of the phoropter/refractor housing, along the support arm (which can allow for the phoropter/refractor to be suspended in front of the patient), and/or down the phoropter/refractor vertical stand (to which the support arm can be affixed). The power cord can be plugged into a conventional power outlet, such as a power outlet located in the wall or floor of the examination room. In certain exemplary embodiments, the speaker and/or speakers can be remotely powered and/or the signal from the electronic display can be communicated wirelessly to the speaker and/or speakers.

In certain exemplary embodiments, the power cord that can connect to the electronic display and audio system can follow the same path as that just described but can plug into a housing that can be capable of receiving electrical plugs located on the stand of the phoropter/refractor. In certain exemplary embodiments, when a battery, batteries, fuel cell, and/or fuel cells are used the power source can be self-contained within or near the electronic display means. In this case, there need not be a power connection means back to the phoropter refractor stand.

Certain exemplary embodiments of the eye examination near point electronic display system can be capable of remote download of information and/or images, which can be provided via a remote source at any time. The remote source can download such information and/or images to any of a plurality of electronic display systems. The plurality of electronic display systems can be those located within one office location and/or numerous separate office locations of one owner and/or numerous owners. These locations can be anywhere, such as within one city or town, one county or counties, and/or spread over numerous states and/or countries, and/or geographical areas, etc.

The eye examination near point electronic display system can utilize a reflective display via which light that normally would illuminate a near point card (or light from another source) can be directed towards the reflective display. The eye examination near point electronic display system can include a backlit display, or transmissive display, such as an LCD display. The eye examination near point electronic display system can use an electronic display that is comprises OLEDs and/or other emissive display technology.

By affixing the display system to the near point rod, which can be affixed to the phoropter/refractor, following the final step or near final step of the eye examination procedure (e.g., when the eye doctor/eye care professional finishes measuring for the near point prescription of the patient), the display system can be (but does not have to be) used for displaying the near point letters or object used in part for establishing the near point corrective lens for the patient. In the case of certain eye disease, such as by way of example only, macular degeneration, the display system can display test patterns capable of detecting macular degeneration and/or measuring its progression.

Once the near point corrective lens or add power is established for the patient, the eye examination near point electronic display system then can be used seamlessly to provide the patient visual information (with or without sound) as to the (by way of example only) type of lens, lens style, material, and/or lens treatments, eye glass frame, eye treatment, surgical procedure, medication, and/or contact lens solution, etc. that the eye doctor/eye care professional recommends. Given that the video, and in most but not all cases, audio, can be prepared and/or approved in advance, the message that is communicated can be accurate and/or consistent.

Upon finishing the eye examination and/or upon the eye doctor/eye care professional determining what he or she feels the best lens solution (by way of example only: lens, lens style, material, and/or lens treatments, eye glass frame, eye treatment, surgical procedure, medication, and/or contact lens solution, etc.) for the patient, the eye doctor/eye care provider then can select the desired information that can provide the proper recommendation to the patient on behalf of the eye doctor or eye care provider. This information can be communicated to the patient by way of video and/or audio with little chance of mistake very efficiently even with the eye doctor/eye care provider not present in the room. For example, the desired information can be communicated to the patient as the patient views the display while looking through the final near point lens correction as selected by the eye doctor/eye care provider in the phoropter/refractor.

Given the current general increase in the population seeking eye care and the 3rd party insurance carriers who pay for eye care services at a discounted rate, many eye care professionals (ECPs) are having to see more patients during a given time period to stay even financially. There can be a need to assist these ECPs so that they can provide quality care in less time. Given the pressure on their time, they often delegate certain functions down-stream as opposed to taking care of it themselves. By way of example only, more and more eye doctors are delegating the refracting portion of the eye exam to technicians. In too many cases, the eye doctor is utilized to check eye health, double check the results of the refracting technician, and then give advice and/or recommendations to the patient as to the state of their eye health and/or their need for corrective eyeglasses should they be needed or should they need to be changed. In the past, the eye doctor would have been with the patient throughout the examination from the beginning to the end. But today more and more eye doctors (not all) begin with checking the health of the patient's eyes, delegate the refraction to a refracting technician, and while the refracting technician is refracting the patient, the eye doctor is off working on another patient. Typically, upon the refracting technician completing the eye refraction portion of the eye examination, the eye doctor will once again interact with the patient. It is during this period of time that the eye doctor often will give the patient advice and/or a prescription of eye glasses if needed. But once again, due to the time constraints that are usually self-imposed but created by having to see more patients within the same period of time to stay even financially, the time period for such advice often has been reduced by the ECP. If the eye doctor is with the patient throughout the entire eye examination, including the eye refraction portion, once again this period of time allocated for the entire eye examination portion, including that of giving advice and counsel, also can be constricted as compared to the amount of time given 10 years ago.

Therefore, there can be a need to help the ECP be more efficient, render care at a consistently high level, and/or give advice of a quality that is consistent with the ECPs desire. Certain exemplary embodiments can provide a method for improving the efficiency of time for the ECP while maintaining very consistent high quality advice for the patient.

Certain exemplary embodiments can provide an electronic display device that can be affixed to the near point rod of the phoropter/refractor, attached to the stand holding the phoropter/refractor, attached to an arm that is affixed to the wall or counter next to the phoropter/refractor, and/or that can be detached and/or held by the patient, set on the patient's legs, and/or set upon a surface. Certain exemplary embodiments can comprise re-chargeable batteries. While the electronic display device is attached to the near point rod, attached to the stand holding the phoropter/refractor, attached to an arm that is affixed to the wall or counter next to the phoropter/refractor, any one or combination of which can comprise a wire and/or connectivity for providing electrical power, the electronic display device can be charging. The ECP can provide a message to be communicated on the display while the patient is looking through the near point lens or lenses within the phoropter/refractor. Should the ECP desire to detach the electronic display device from the near point rod, the electronic display device can easily be detached and then viewed by the patient. Various electronic messages can be stored after being downloaded and/or transferred from a source and/or via a mechanism such as, by way of example only, a satellite, Wi-Fi, flash card, computer network, cable, cell tower, wireless communication, phone line etc.

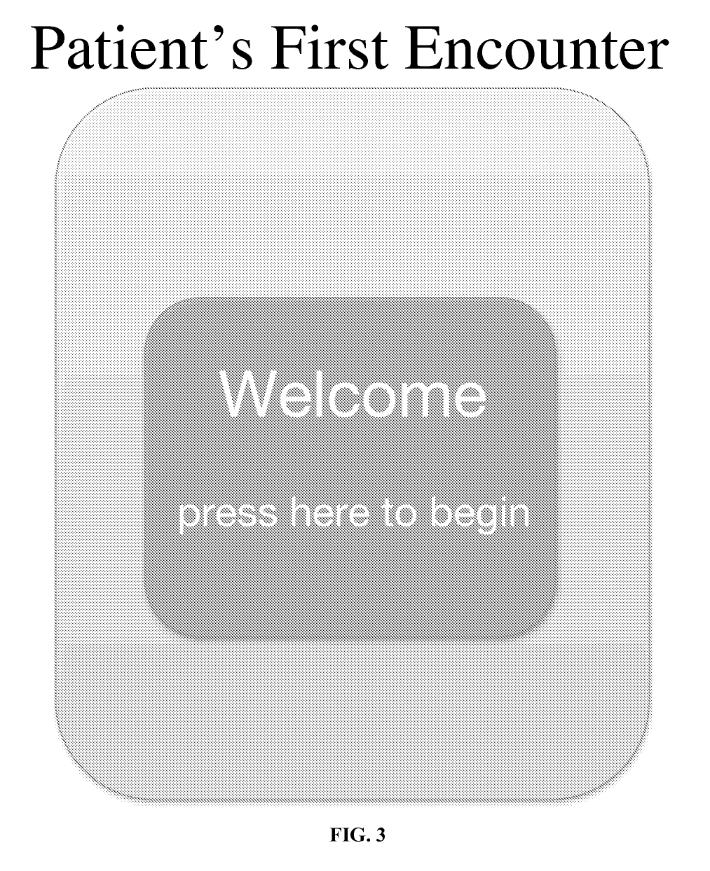

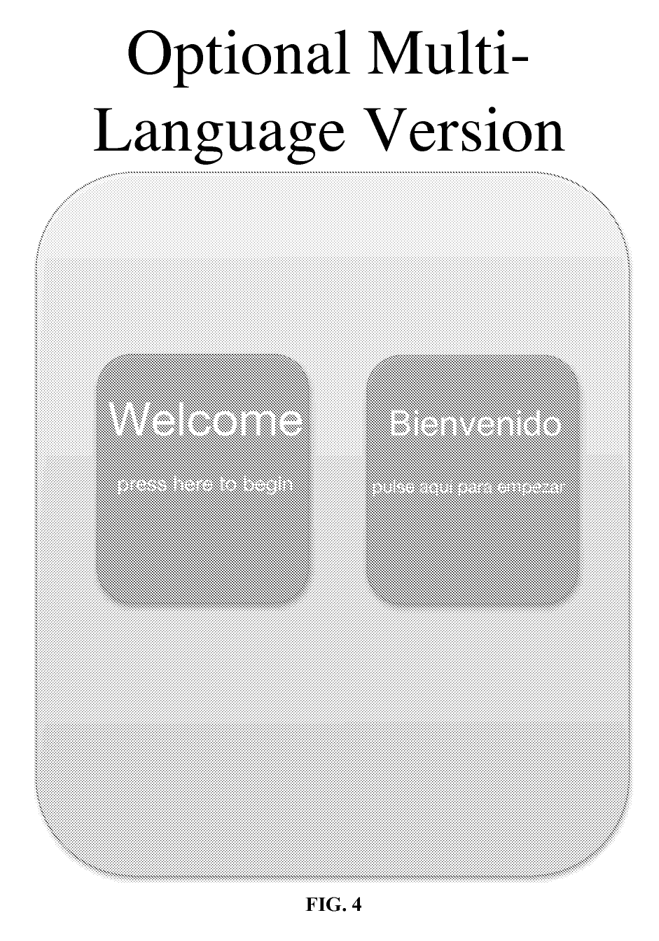

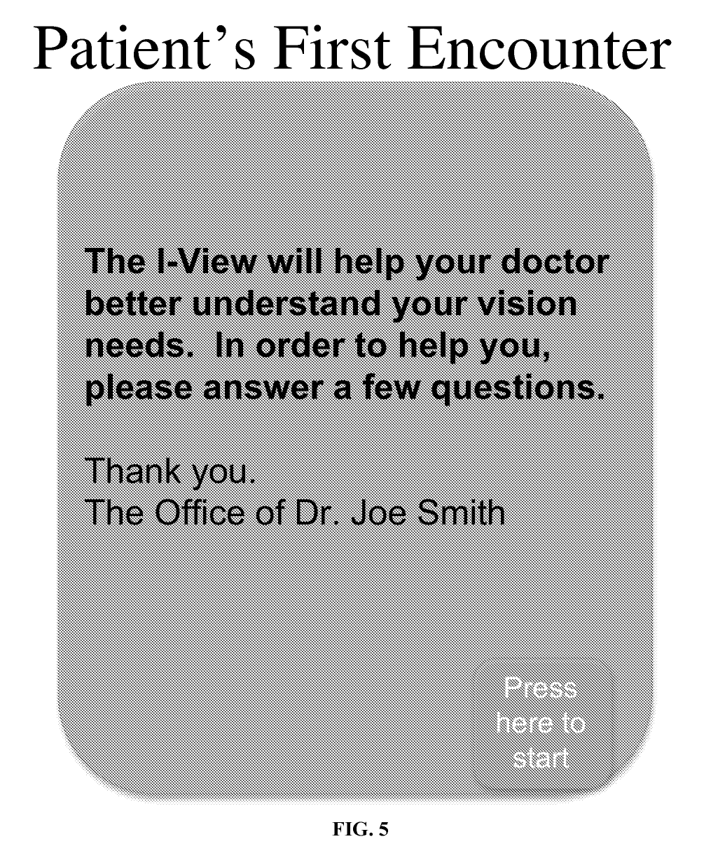

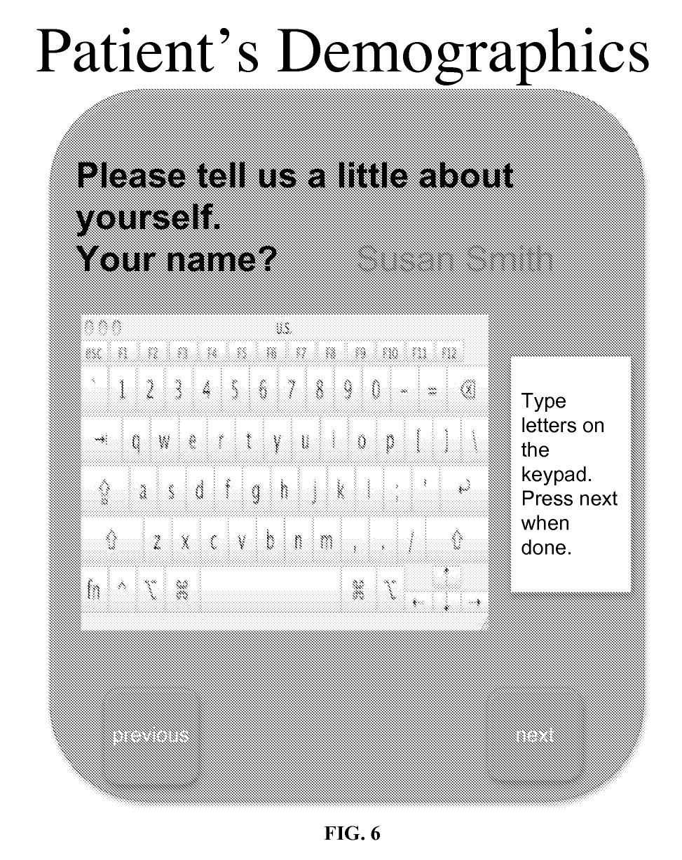

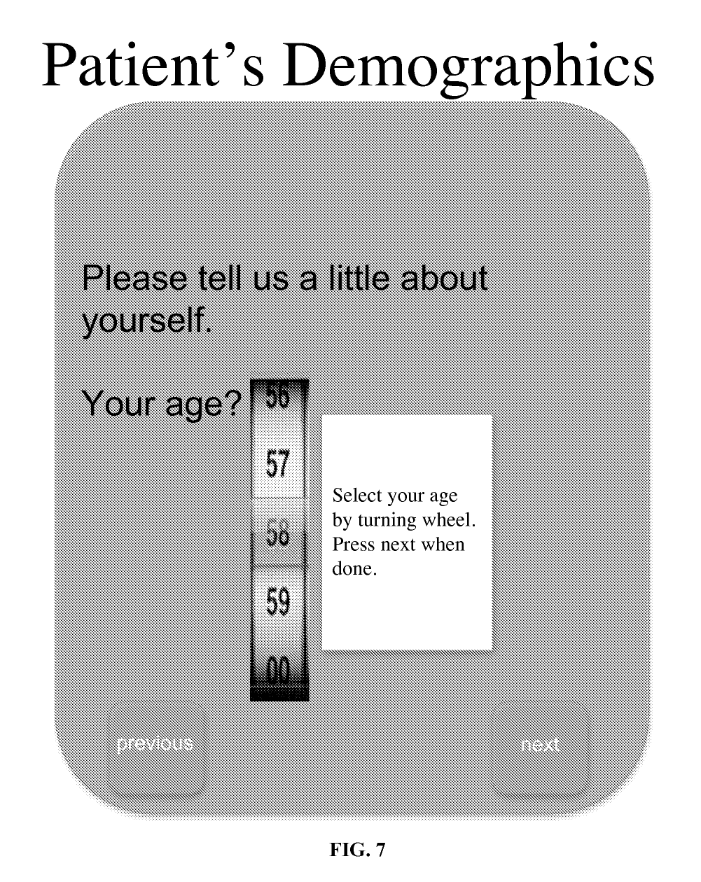

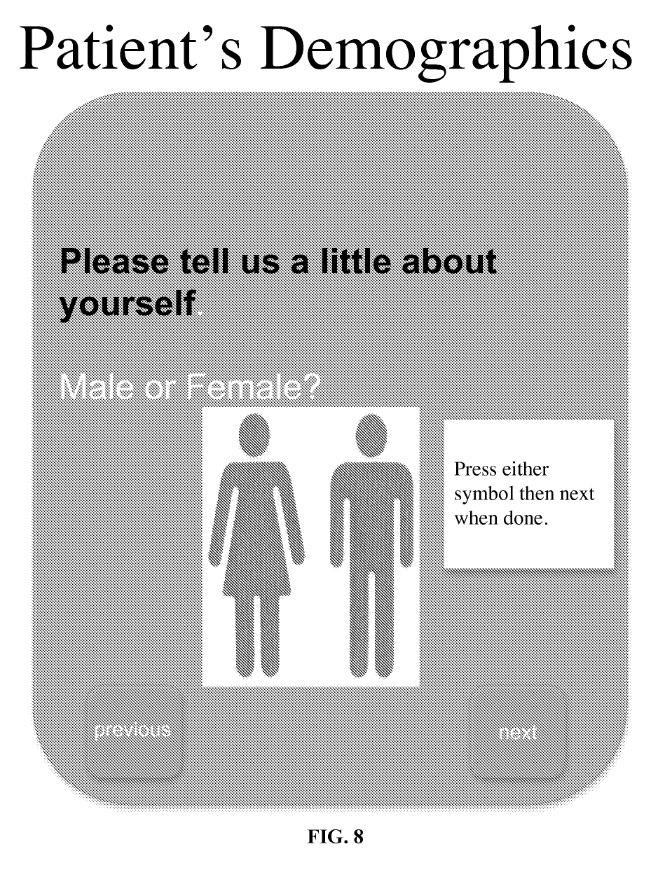

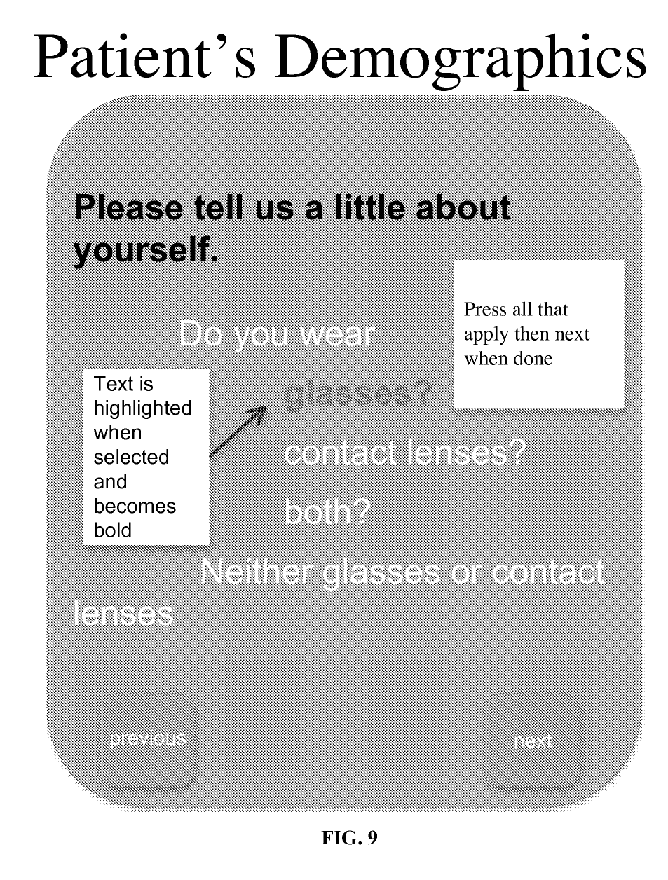

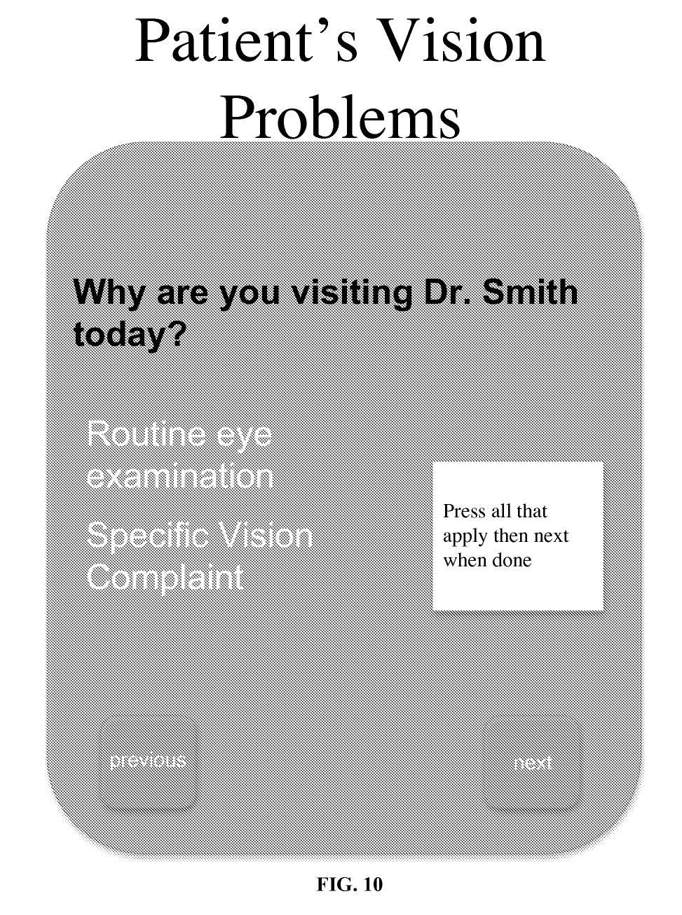

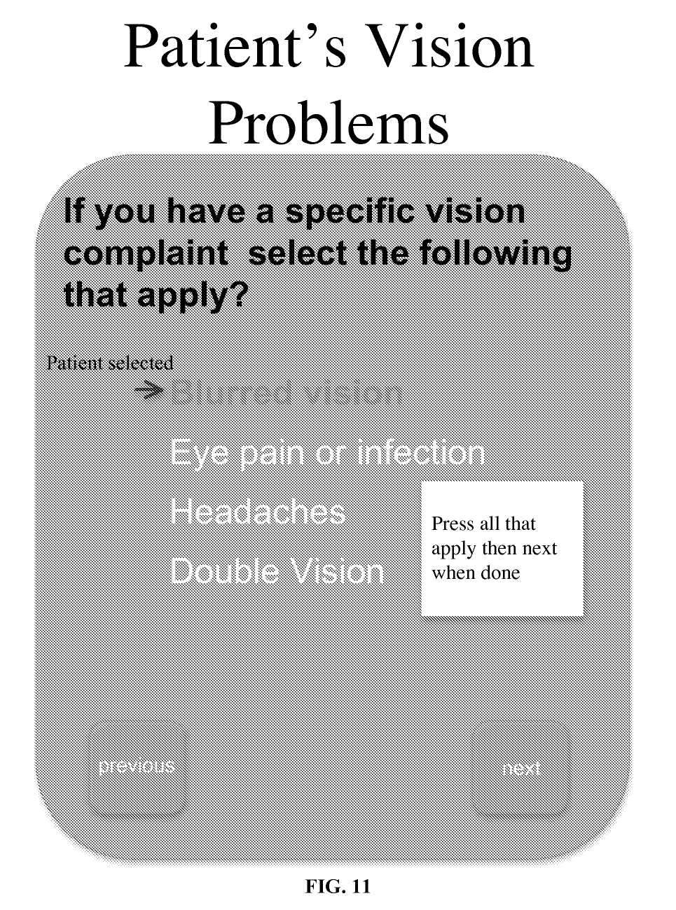

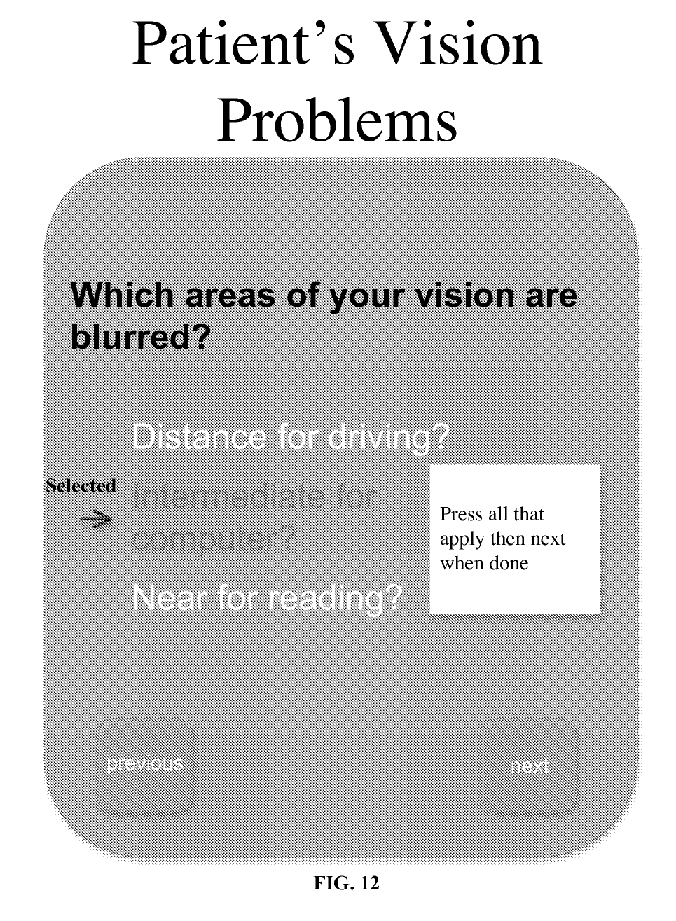

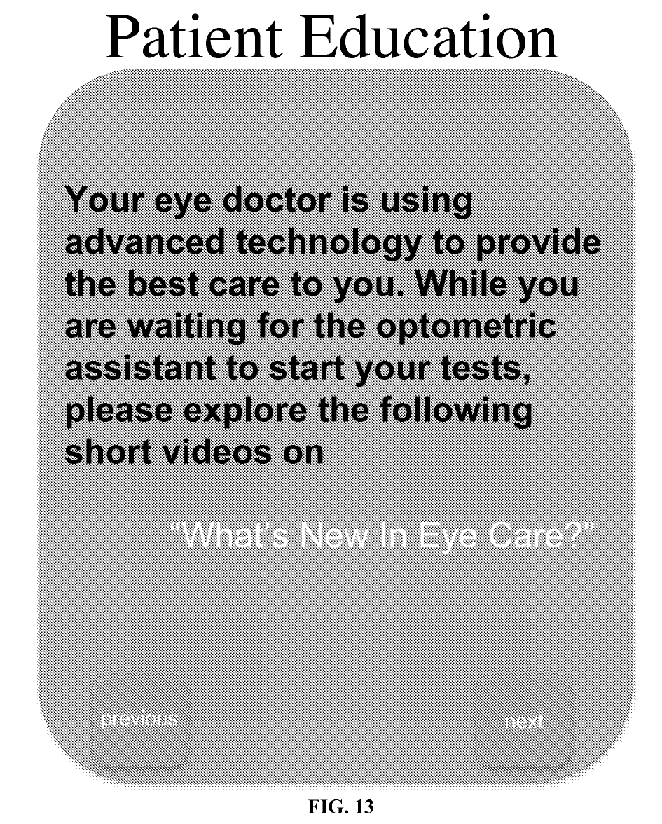

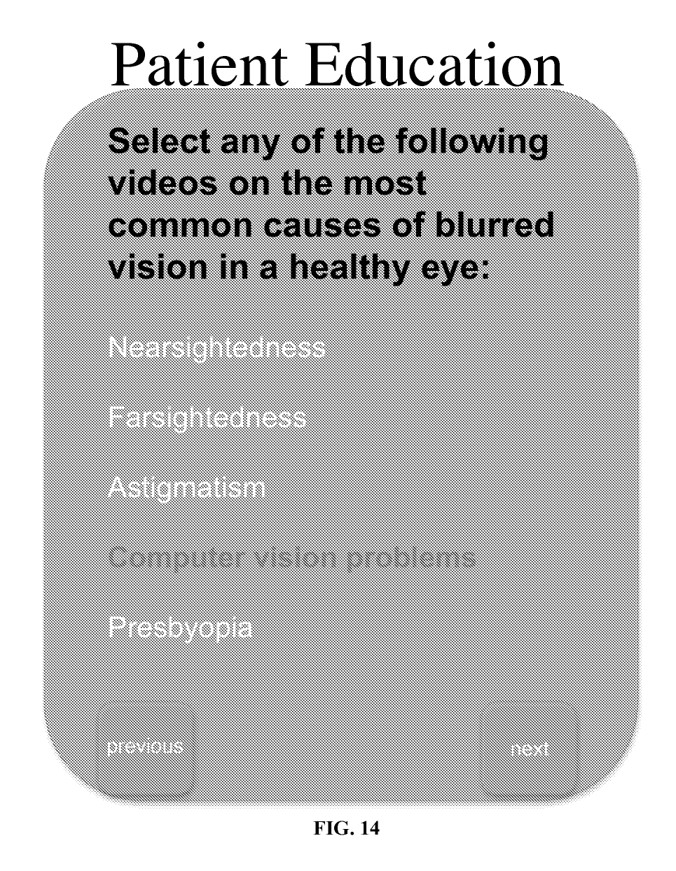

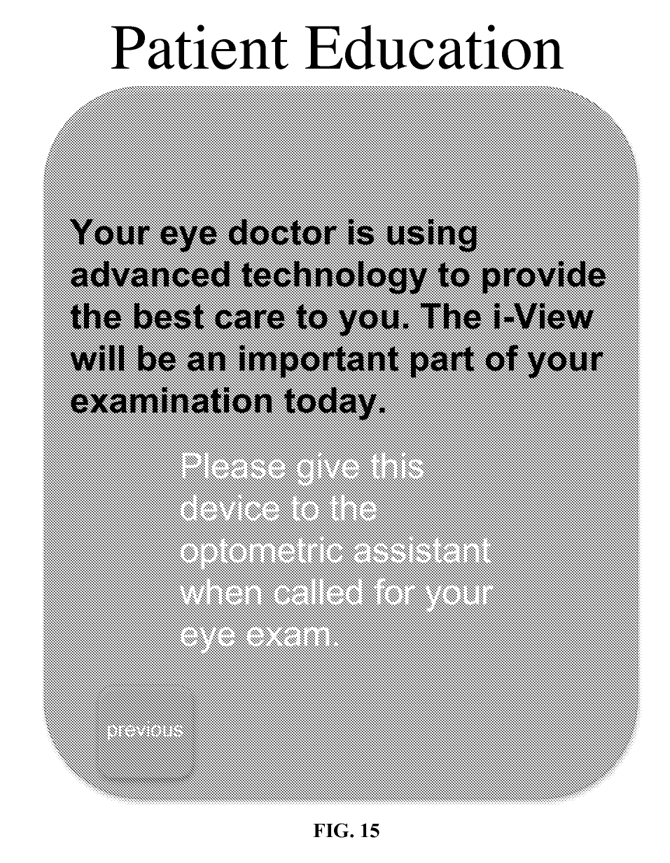

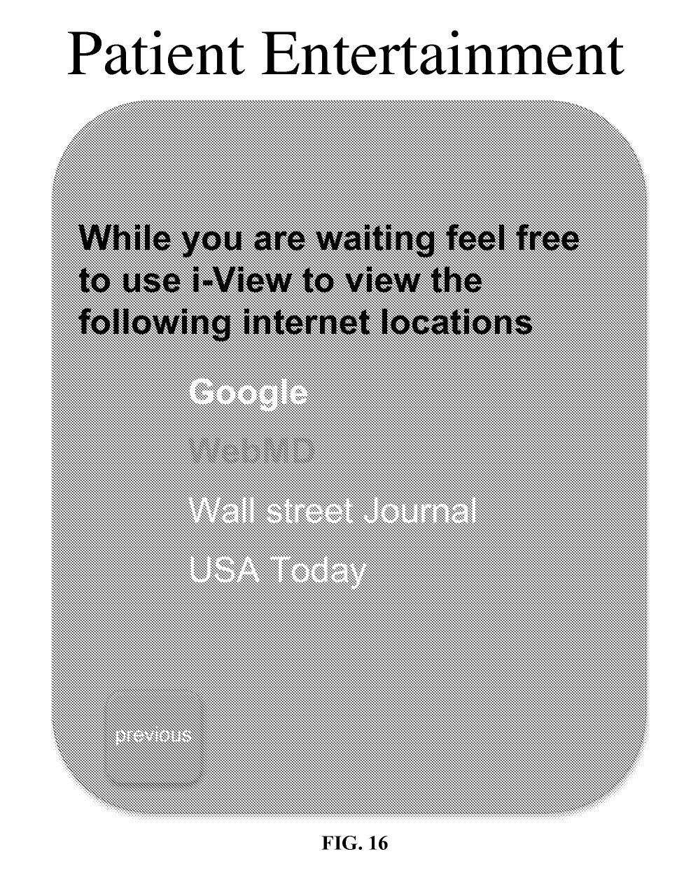

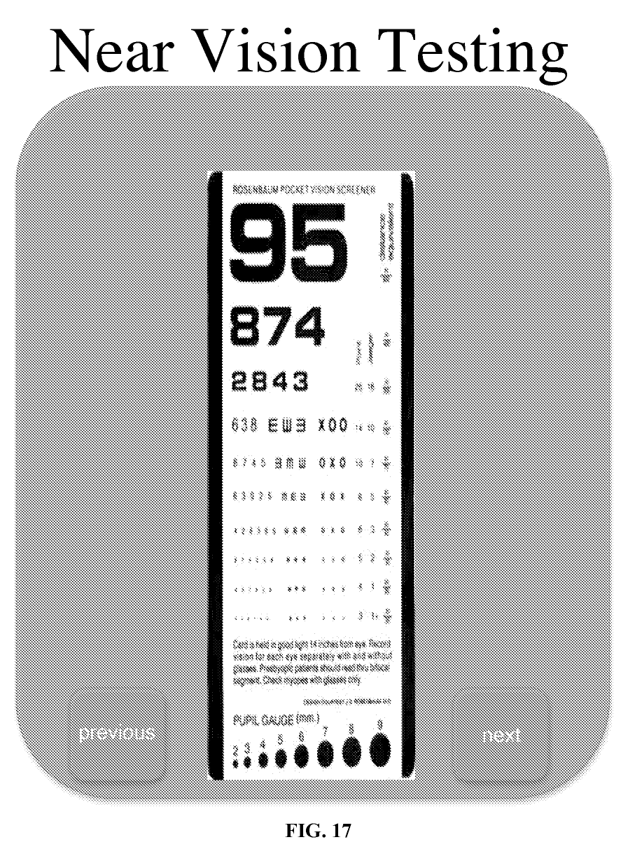

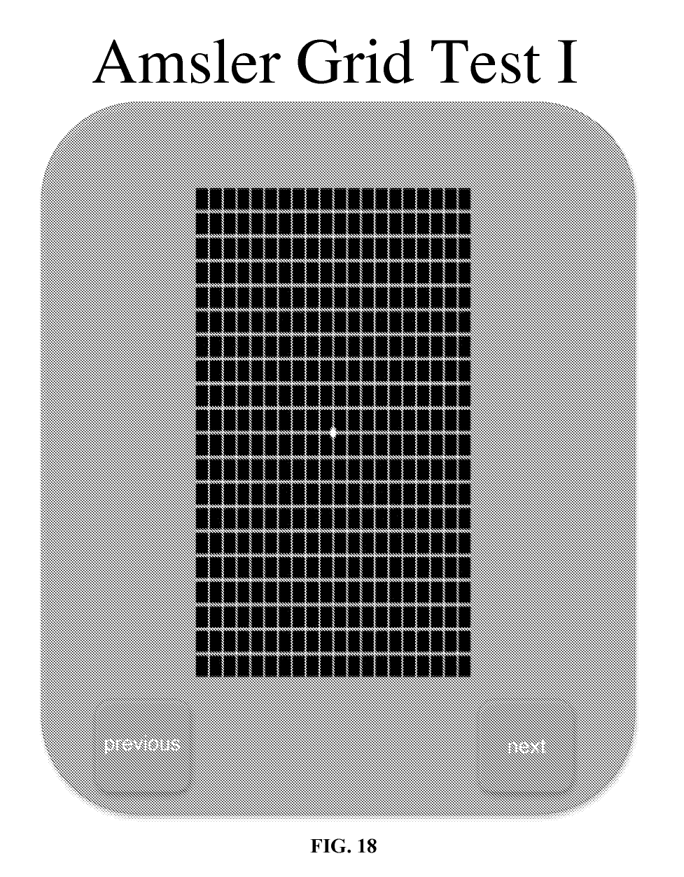

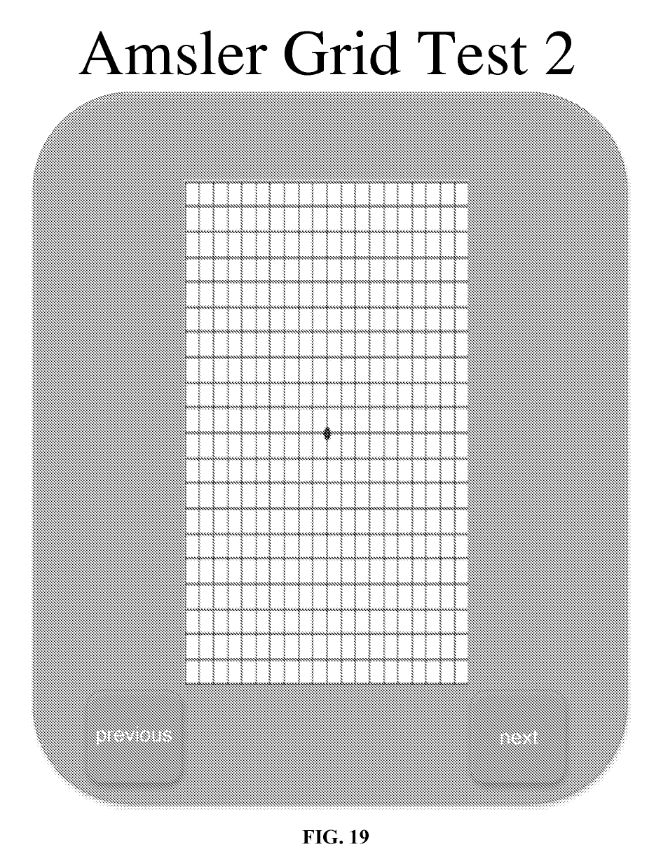

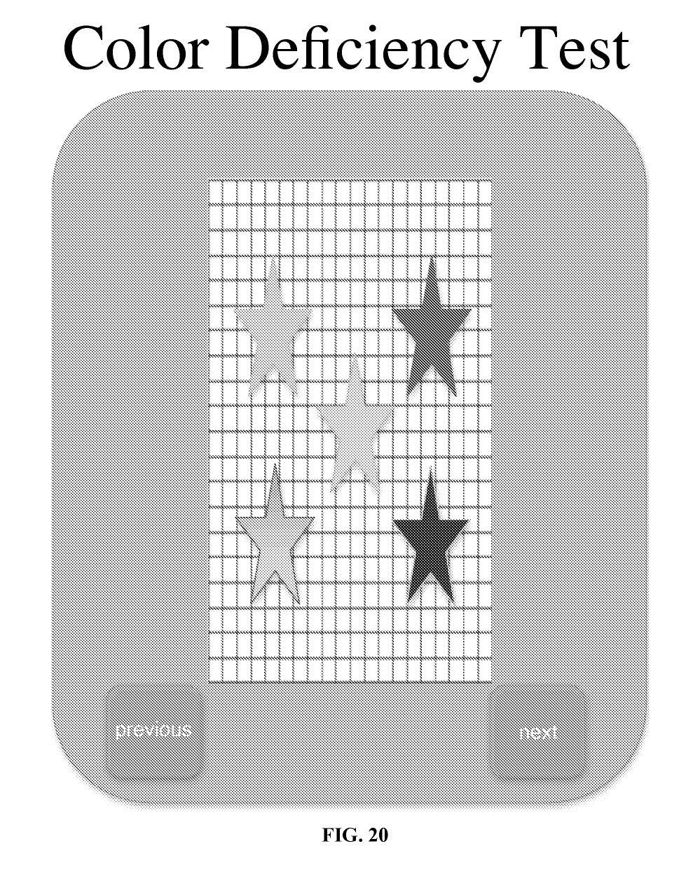

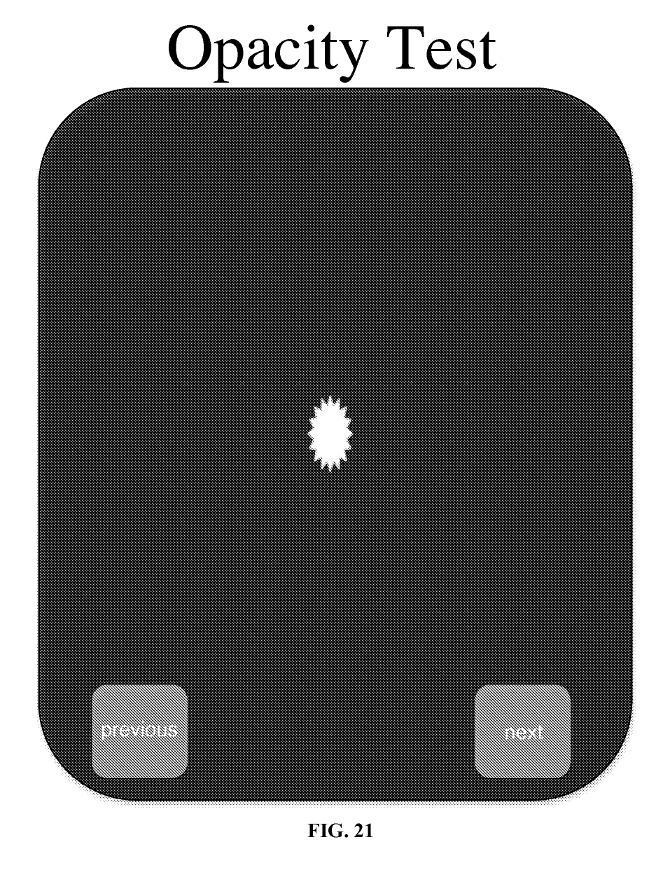

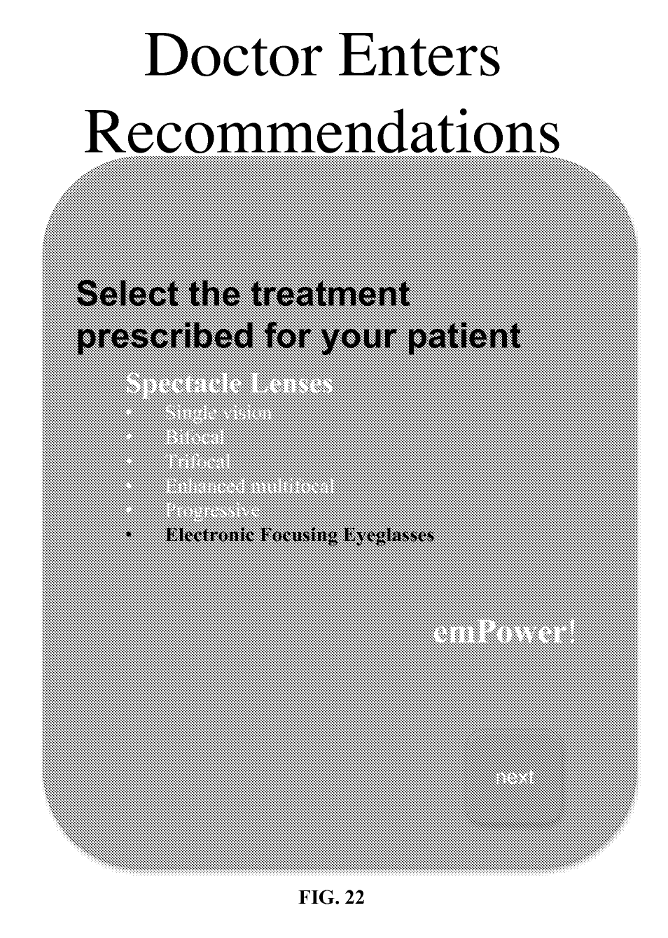

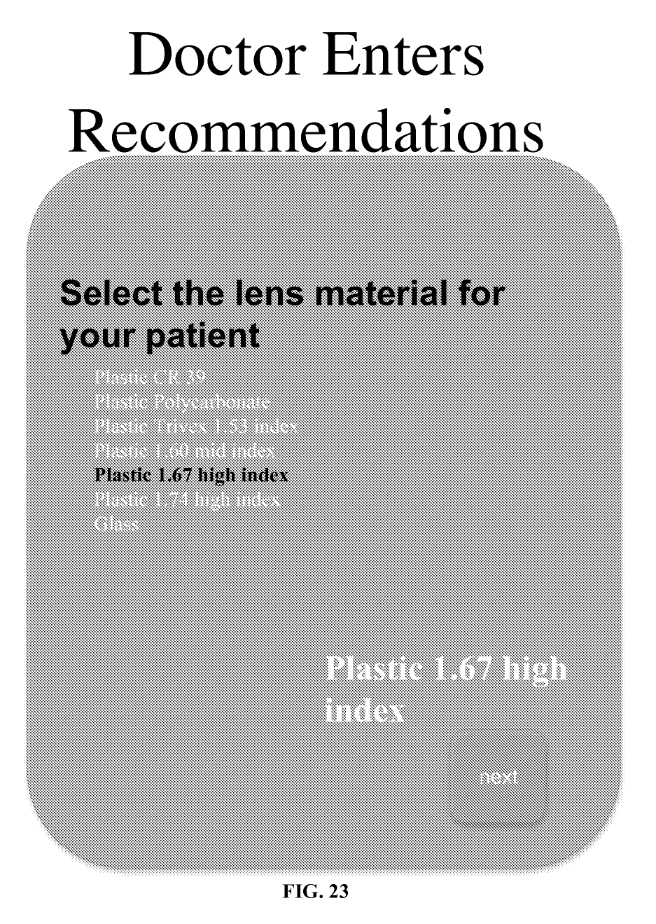

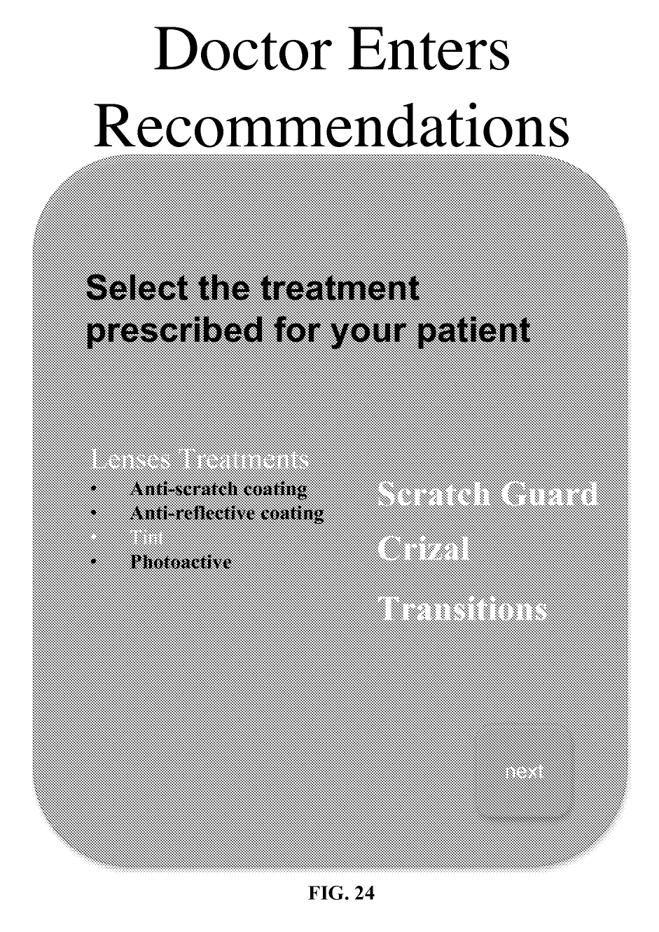

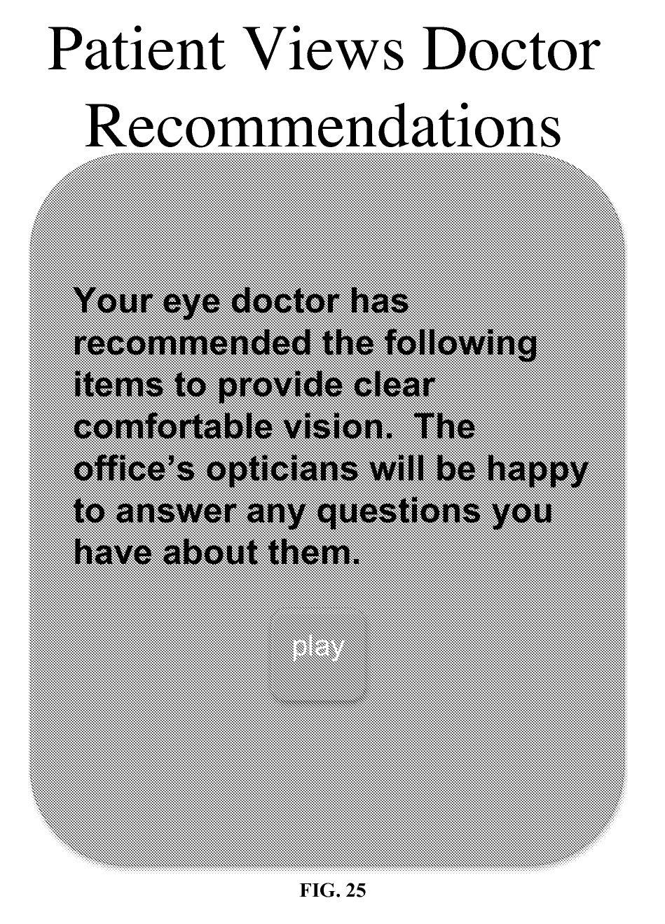

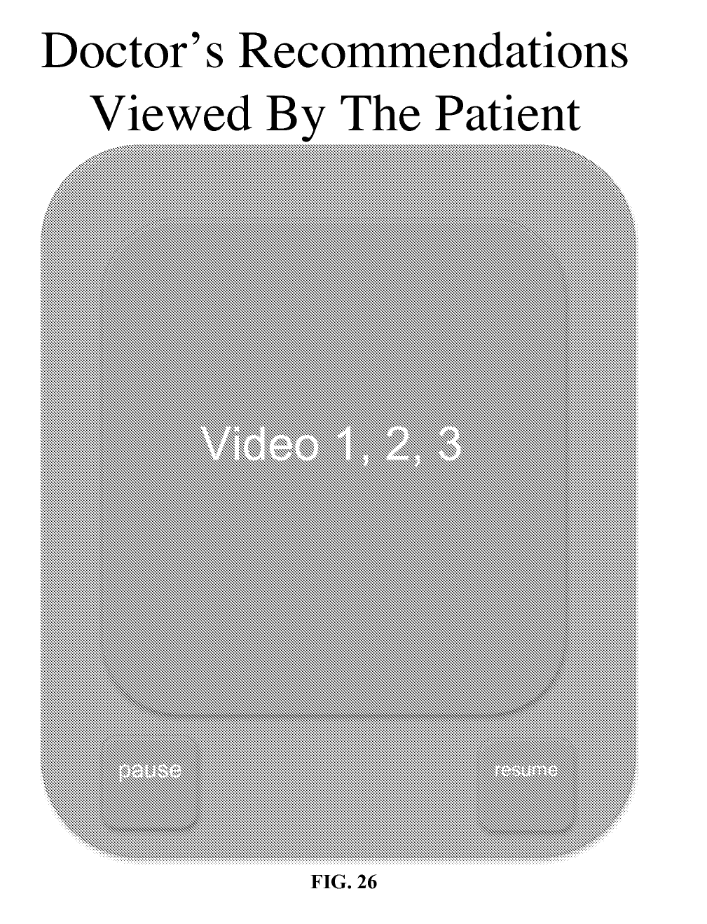

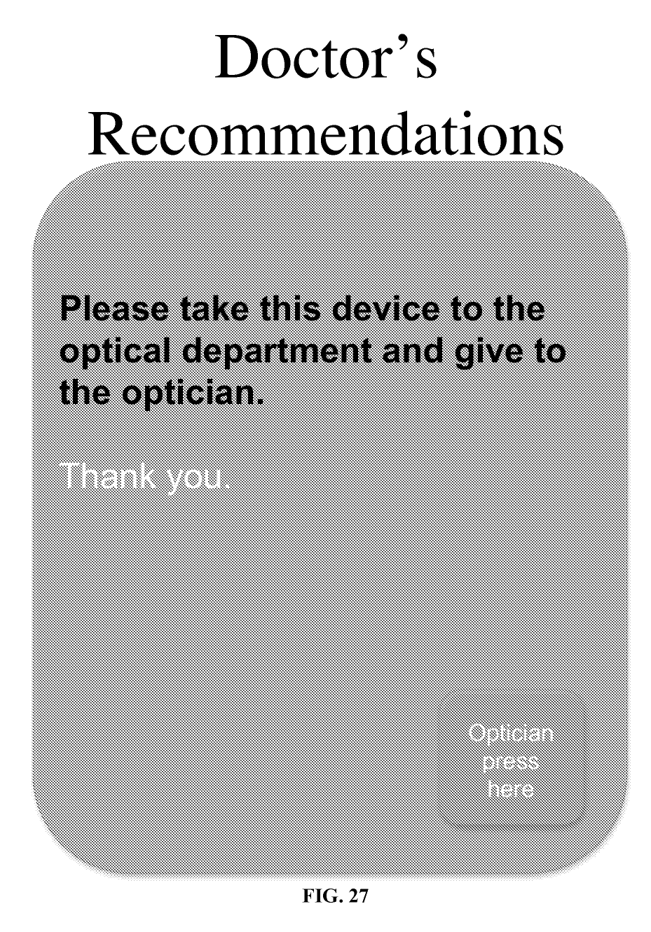

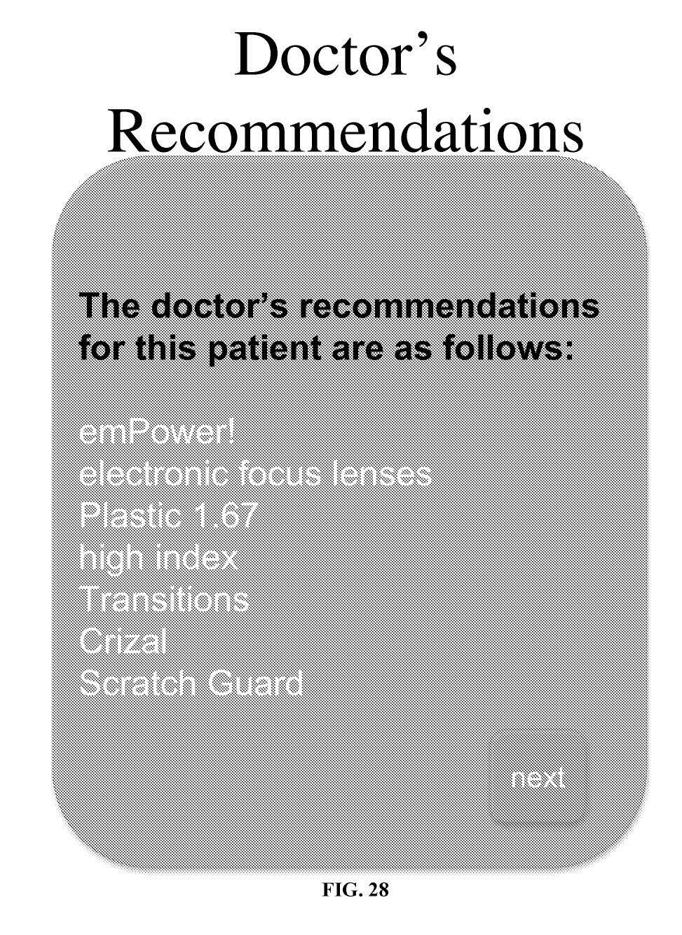

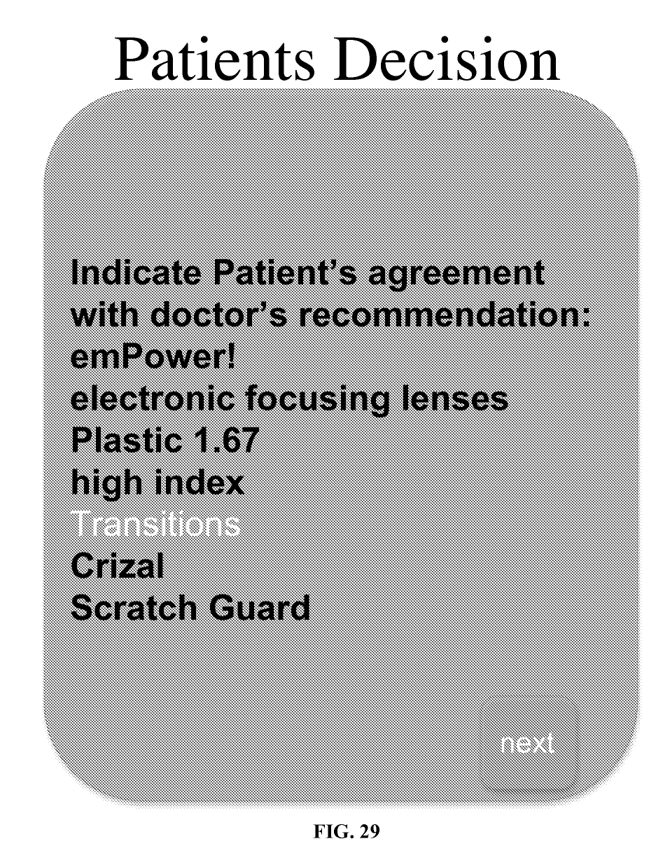

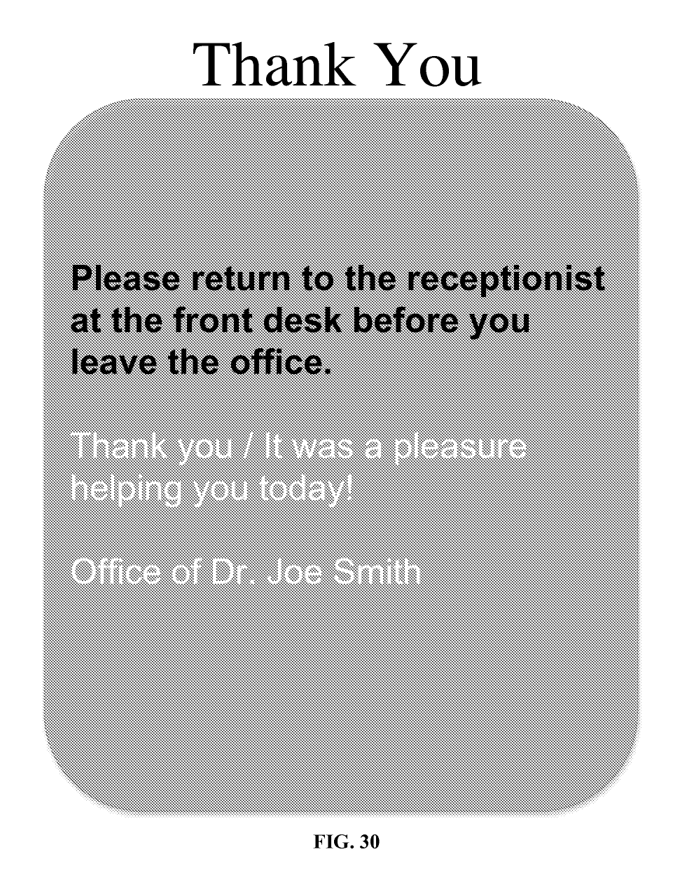

As shown in FIGS. 3-30, certain exemplary embodiments can provide one or more user interfaces that can, for example, allow for the customary and/or traditional near point chart that is typically supplied by the maker of the phoropter/refractor being used (or one that is of the ECP's liking) to be affixed to the back of the display. The electronic display device can be rotated while affixed to the near point rod. This can allow for the ECP to utilize the same near point chart that the ECP is accustomed to when performing the eye refractive portion of the eye examination. The electronic display device can provide the appropriate near point image and/or images (static, flash, or dynamic/motion) required to test near vision of the patient and/or can provide for health testing, such as, by way of example only, displaying an Amsler Grid to check for macular degeneration.

The electronic display device can comprise a controller that can allow for the ECP to program in real time for each patient the recommendation that the ECP recommends. The controller can be affixed to the display or can be remote from the display. The controller can allow the ECP to select various menu options and/or messages that the ECP would like to communicate, by text, audio, and/or video, to the patient and/or in the order that the ECP would like those items to be communicated. By way of example only, should the ECP desire to communicate that the ECP is recommending that the patient wear:
1) light weight plastic lenses;
2) that are of a high index 1.67 material;
3) being Essilor Definity progressive addition lenses to correct for the patient's vision at distances of far, near, and in between;
4) comprising a UV inhibitor for UV blocking protection;
5) that change their tint with exposure to sun light such as Transitions photochromatic lenses; and
6) have Essilor's Crizal anti-reflective coating for improving night vision performance;
   the ECP can select those choices from a menu of options and/or messages and can do so in the numerical order as just shown. Such a menu can be presented by software running on the electronic display device, which can be a touch screen display, and/or can be remotely provided via a remote controller and/or communicated by wire and/or wirelessly to the electronic display device. The menu can be accessed by way of any all known means; by way of example only, manual switches, manual buttons, touch screen, computer mouse, wireless remote control, and/or auditory commands using voice recognition software.

The electronic display device can communicate directly or indirectly with the optical dispensary in a wired and/or wireless manner, and/or carried into the optical dispensary by the patient and/or technician. Such communication(s) can provide the optical dispensary the pertinent ECP recommendations for the patient (potentially including the lens prescription if indicated by the ECP). By communicating directly with the optical dispensary, the ECP can avoid the possibility that the recommendation will be altered, misinterpreted, and/or lost.

Businesses involving healthcare professionals such as, but not limited to, dentists, medical doctors, podiatrist, vision care professionals (optometrists, ophthalmologists, opticians), pharmacists, pharmacologists, chiropractors, medical technologists, and/or others, have become very regulated business and/or professions in the United States and other countries around the world. In many cases, but not all, certain regulations can prohibit and/or make it very difficult for a healthcare professional to recommend a product and/or treatment that would help a patient and/or patients if it is perceived that there is a financial reward (or kick back) being offered to the healthcare professional for recommending and/or selling such a product and/or treatment. In the United States, such regulations often are enforced by the FTC (Federal Trade Commission).

Companies offering a product (such as a pharmaceutical, device, and/or treatment) that might help a patient often must comply with the rules or face serious penalties. In addition, those professions that police themselves often deem it to be unethical for their professionals to engage in promoting a product whereby there is a kickback and/or financial reward offered by the company making the product when sold and/or recommended to a patient, even if that product helps the patient with his or her affliction.

This policy has been put into place ostensibly to protect the public from professionals that would recommend a given product over other competing products only for reasons of financial reward and not because it is in the best interests of the patient. While this policy can be in the best interests of the patient, it can inhibit the business and/or marketing policies and/or approaches of companies wanting to sell and/or market a product and/or products to healthcare professionals. Therefore there is a need for an approach that can allow and/or motivate the healthcare professional to recommend a product and/or products to a patient and/or patients without receiving what would be perceived to be a financial kickback directly and/or indirectly from the company manufacturing, distributing, and/or marketing the product and/or products. In certain exemplary embodiments, such motivation (e.g., legally permitted incentive) can be provided by the growing of the healthcare professional's practice by way of improving the quality of the recommendation to the patient and/or the efficiency in which it is made.

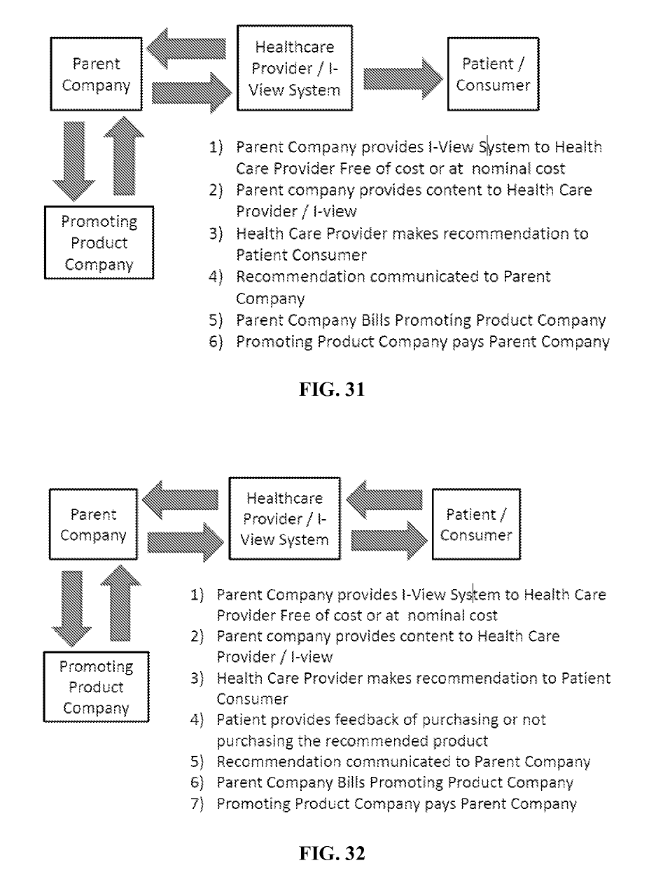

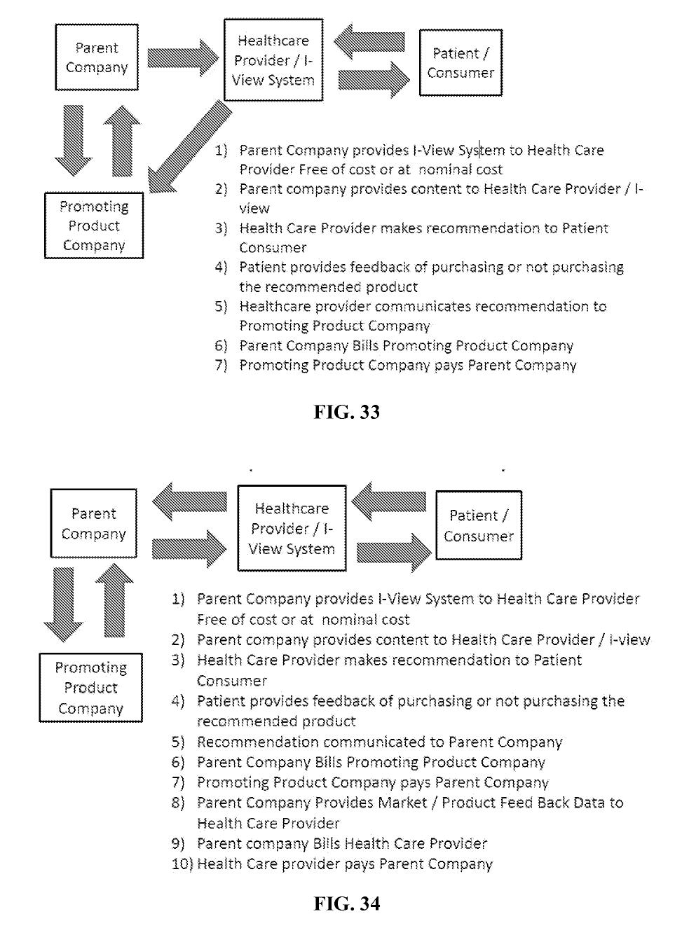

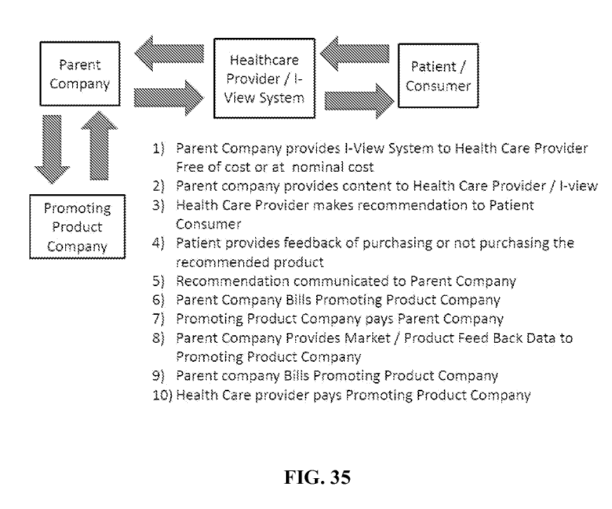

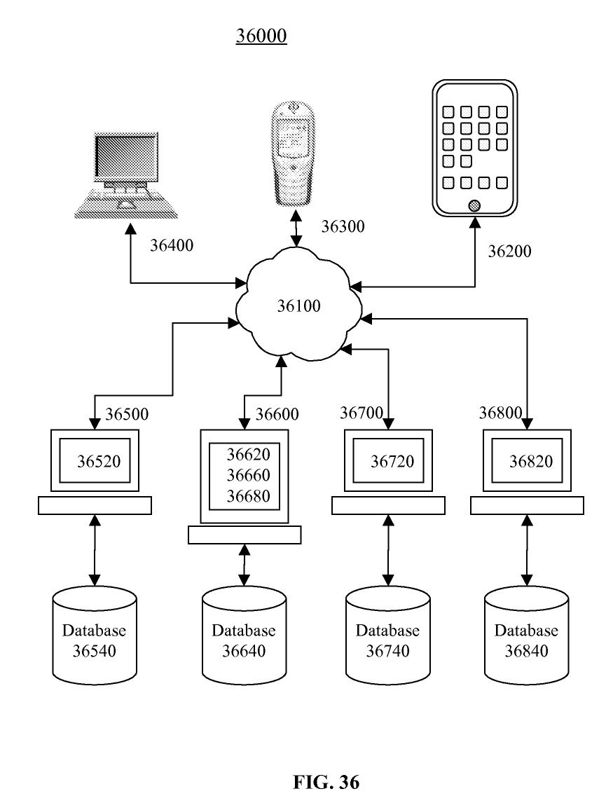

FIGS. 31-35 are flow charts of one or more exemplary embodiments of one or more approaches and/or methods whereby a healthcare professional can be provided with a communication system that can allow for the healthcare professional to better inform, recommend, and/or educate his/her patients of various diseases and/or products. Certain exemplary embodiments of this communication system, sometimes referred to herein as the “I-View System”, can be of any communicative form, such as a communication display (electronic and/or non-electronic), text (printed and/or electronic), and/or speaker.

Certain exemplary embodiments can provide for an entity, such as a healthcare information intermediary and/or an eyecare information intermediary (called “the parent company” in FIGS. 31-35) that can create and/or provide the content that is shown on one or more I-View Systems. The parent company can contract with an entity, such as a healthcare product company and/or an eyecare product company (called a product company in FIGS. 31-35) and/or companies that desire to promote and/or communicate the product company's product and/or products via the I-View System. For most, if not all products promoted and/or communicated on the I-View System, there can be multiple competing products from different manufacturers. The healthcare provider can choose which of the competing products and/or medical procedures he or she recommends to the patient.

The content can be provided directly from the product company to the I-View System and/or the parent company can approve and/or control distribution of this content. The I-View System can allow for the healthcare provider to recommend a product or products to a patient whereby his or her recommendation can be communicated on or by the I-View System. The recommendation can be communicated, by way of example only, wireless, wired, electronically (by way of the web or internet), text, or copy to the parent company, whereby the parent company then can bill the product company and/or the product company can pay such bill to the parent company. The recommendation can be communicated to the product company and to the parent company whereby the parent company then can bill the product company and/or the product company can pay such bill to the parent company. Thus the I-View System can provide timely feedback (which in some cases can be real time feedback) to the parent company and/or the product company.

The product company can pay a different fee to the parent company for various combinations of data. By way of example only, should the healthcare provider recommend product X then there might be a fee paid from the product company to the parent company of (by way of example only) $1.00. However, should the patient select the product that was recommended by the healthcare provider then the fee paid might be (by way of example only) $3.00. The I-View System can allow for communicating the healthcare provider's recommendation and/or whether or not the recommendation was adhered to by the patient (by way of the patient purchasing such a product) to the parent company and/or to the product company. Thus the I-View System can allow for the product company to know when their product is being recommended and/or when their product is being both recommended and purchased by the patient. Certain exemplary embodiments can allow one or more product companies to provide content directly and/or indirectly via each I-View System.

In this manner, the healthcare provider need not be paid a kickback for recommending the product to the patient. The patient can win as he or she can receive better information and/or be better informed. The healthcare provider can win as he or she can provide the patient with a better quality of care. The parent company can win as the parent company can receive revenue and/or profits from providing recommendations of the healthcare provider to the relevant product companies. Each product company can win as that product company legally can promote its products by way of the healthcare provider's recommendation to the patient and/or receive measurable data indicating that such a recommendation was made and/or that a recommendation was adhered to and/or purchased by the patient. In certain exemplary embodiments, the I-View System can be purchased by the healthcare provider from the parent company or another company, can be provided to the healthcare provider free of charge by the parent company or another company, and/or can be purchased from the parent company with the understanding that such purchase price will be credited back based upon the healthcare provider's use of the I-View System.

Certain exemplary embodiments can allow and/or provide for the parent company to use data that can be provided and/or communicated to it from the I-View System as metrics and/or market information data that can be sold and/or given to the healthcare provider to help the healthcare provider in growing his or her practice, and/or to the product company to allow the product company to better market and/or design its product and/or products.

Although certain exemplary embodiments have been described in the context of healthcare providers, the described approaches are not necessary limited to healthcare, and potentially can be utilized in any profession and/or business that would benefit financially from such an approach.

FIG. 36 is a block diagram of an exemplary embodiment of a system 36000, which can comprise one or more mobile information devices 36200, 36300, via which a patient and/or consumer can receive encrypted and/or unencrypted information, such as product information, a test, and/or a recommendation, etc., and/or can enter and/or provide encrypted and/or unencrypted information, such as personal information, a purchase decision, test responses, product feedback, etc. Mobile information devices 36200, 36300 can be communicatively coupled to one or more inter-connected communication networks 36100, to which can be communicatively coupled one or more service provider information devices 36400, 36500, that can receive encrypted and/or unencrypted information, such as product information, test responses and/or results, customer and/or patient purchase decisions, and/or customer and/or patient feedback, etc., and/or can enter and/or provide encrypted and/or unencrypted information, such as business information, recommendations for customers and/or patients, feedback regarding products, etc.

Communicatively coupled to network 36100 can be one or more intermediary information devices 36600, which can receive encrypted and/or unencrypted information, such as product information, business information, recommendations for customers and/or patients, customer and/or patient purchase decisions, patient test responses and/or results, product feedback, etc., can report and/or provide encrypted and/or unencrypted information, such as product information, recommendations for customers and/or patients, customer and/or patient purchase decisions, patient test responses and/or results, and/or feedback regarding products, etc., and/or can request compensation, such as for reporting and/or providing information, such as any of that just described.

For example, a particular intermediary information device 36600 can receive encrypted and/or unencrypted information regarding each of multiple products, each product provided by a corresponding one of multiple product providers. Consequently, via an information device, each product provider can provide the intermediary with encrypted and/or unencrypted information regarding each of multiple products. As another example, a particular intermediary information device 36600 can receive encrypted and/or unencrypted information regarding each of multiple businesses, each business associated with one or more service providers. Thus, via an information device, each service provider and/or their business can provide the intermediary with encrypted and/or unencrypted information about that service provider and/or business. As still another example, a particular intermediary information device 36600 can receive encrypted and/or unencrypted information regarding each of multiple recommendations, potentially at least a sub-set of those recommendations for customers and/or patients of a particular business and/or service provider. Therefore, via an information device, each service provider and/or business can provide any portion of its recommendations, potentially “sanitized” to remove patient-identifying information and/or private patient information, to the intermediary. As yet another example, a particular intermediary information device 36600 can receive encrypted and/or unencrypted information regarding each of multiple purchase decisions, each purchase decision associated with a given customer and/or patient, at least a sub-set of purchase decisions potentially associated with a given service provider. Hence, via an information device, each service provider, business, and/or its customers and/or patients can provide the intermediary with any portion of the purchase decisions of those customers and/or patients.

Generally, a particular intermediary information device 36600 can decrypt, transform, sanitize, sort, summarize, aggregate, segregate, and/or encrypt received information, as desired, prior to reporting and/or providing that encrypted and/or unencrypted information to others. For example, if it receives information sufficient to uniquely identify a particular patient, the intermediary information device 36600 can remove that information prior to providing related information to others that do not have a legal right to receive the removed information. As another example, if it receives information sufficient to uniquely identify a particular service provider and/or business, the intermediary information device 36600 can remove that information prior to providing related information to others. For example, an intermediary can receive, decrypt, sort, transform, encrypt, and/or report information regarding product purchases in a given area, such as a given country, state, county, metropolitan area, city, zip code, etc., but can first sanitize received information to remove information sufficient to identify particular service providers and/or businesses. Alternatively, an intermediary can aggregate, sort, and/or segregate received information by service provider and/or business and/or can include information sufficient to identify particular service providers and/or businesses.

A particular intermediary information device 36600 can report and/or provide, for example, encrypted and/or unencrypted information regarding each of multiple products, to one or more service providers, such as healthcare service providers. Consequently, via an information device, a given service provider can receive encrypted and/or unencrypted information regarding each of multiple products it produces, distributes, and/or markets. As another example, a particular intermediary information device 36600 can report and/or provide encrypted and/or unencrypted information regarding each of multiple businesses and/or service providers to one or more product providers. As still another example, a particular intermediary information device 36600 can report and/or provide encrypted and/or unencrypted information regarding each of multiple recommendations, potentially at least a sub-set of those recommendations for customers and/or patients of a particular business and/or service provider, to one or more product providers. That is, a given product provider potentially can receive a report of recommendations made for each of its products, those recommendations potentially segregated by and/or aggregated by service provider and/or business, those recommendations potentially sanitized to remove private, legally-protected (e.g., HIPAA), patient-sensitive, and/or patient-identifying information, such as information sufficient to uniquely identify a particular patient, and/or potentially sanitized to remove information sufficient to uniquely identify a particular service provider and/or business. As yet another example, a particular intermediary information device 36600 can report and/or provide to one or more product providers, encrypted and/or unencrypted information regarding each of multiple purchase decisions, each purchase decision associated with a given customer and/or patient, at least a sub-set of purchase decisions potentially associated with a given business and/or service provider. That is, a given product provider potentially can receive a report of purchases of its products, potentially segregated by service provider and/or business and/or correlated with recommendations of those products, those purchases and/or recommendations potentially sanitized to remove private, legally-protected (e.g., HIPAA), patient-sensitive, and/or patient-identifying information. As a further example, a particular intermediary information device 36600 can report and/or provide to a given product provider a request for compensation such as for reporting and/or providing encrypted and/or unencrypted information, such as that just described, and/or can receive encrypted and/or unencrypted information (such as a confirmation and/or clearance of payment) regarding such compensation.

Communicatively coupled to network 36100 can be one or more product provider information devices 36700, 36800, which can receive encrypted and/or unencrypted information, such as product recommendations for customers and/or patients, customer and/or patient purchase decisions, and/or feedback regarding products, etc., can provide encrypted and/or unencrypted information, such as product information, and/or can provide compensation, such as compensation for receiving recommendations for customers and/or patients, customer and/or patient purchase decisions, and/or feedback regarding products, etc.

Any information device, such as information device 36600, can comprise a user interface 36620, such as a display, touch screen, keyboard, mouse, keypad, trackball, touchpad, pointing device, stylus, microphone, speaker(s), projector, etc. Running on any information device, such as information device 36600, can be software 36660, which can facilitate any of the functions described herein in association with an information device, I-View System, eyecare display device, etc. For example, any information device, such as information device 36600, can run a database management system 36680, which can manage encrypted and/or unencrypted data stored in a data repository and/or database 36640.

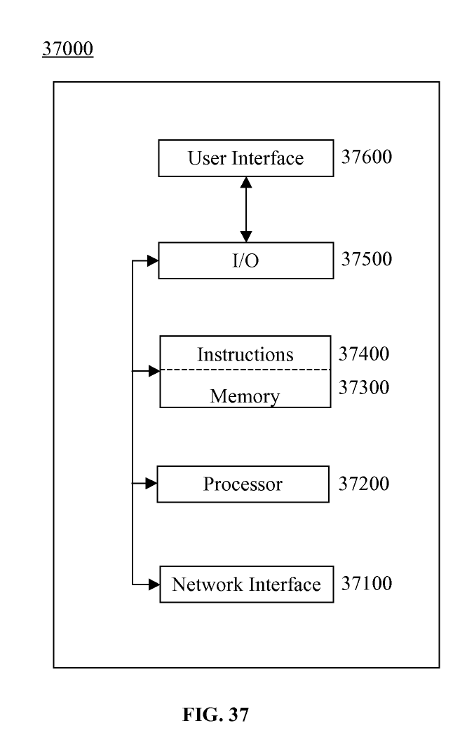

FIG. 37 is a block diagram of an exemplary embodiment of an information device 37000, which in certain operative embodiments can comprise, for example, one or more mobile information devices 36200, 36300, one or more service provider information devices 36400, 36500, one or more intermediary information devices 36600, and/or one or more product provider information devices 36700, 36800 of FIG. 36. Information device 37000 can comprise any of numerous transform circuits, which can be formed via any of numerous communicatively-, electrically-, magnetically-, optically-, fluidically-, and/or mechanically-coupled physical components, such as for example, one or more network interfaces 37100, one or more processors 37200, one or more memories 37300 containing instructions 37400, one or more input/output (I/O) devices 37500, and/or one or more user interfaces 37600 coupled to I/O device 37500, etc.

In certain exemplary embodiments, via one or more user interfaces 37600, such as a graphical user interface, a user can view a rendering of information related to researching, designing, modeling, creating, developing, building, manufacturing, operating, maintaining, storing, marketing, selling, delivering, selecting, specifying, requesting, ordering, receiving, returning, rating, and/or recommending any of the products, services, methods, user interfaces, and/or information described herein.

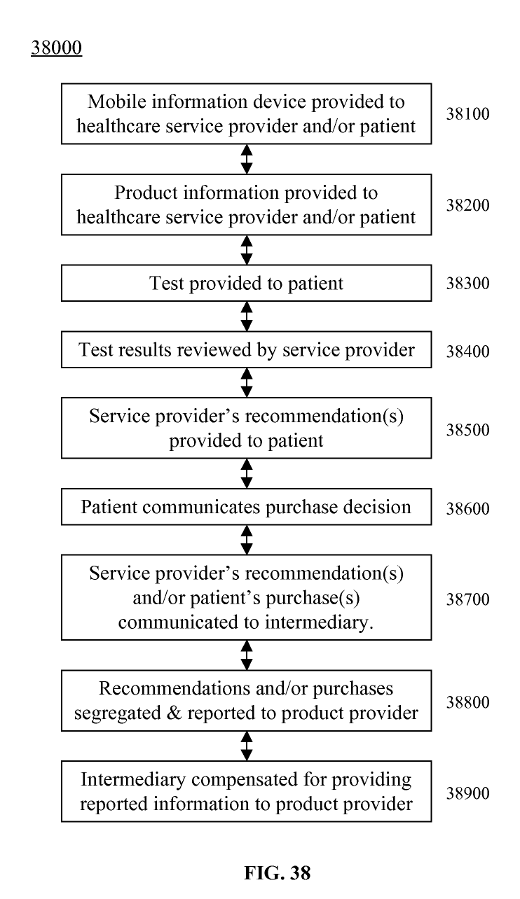

FIG. 38 is a flowchart of an exemplary embodiment of a method 38000. At activity 38100, a mobile information device can be provided to a service provider, such as a healthcare service provider (e.g., an eye care professional), to a patient, and/or to a customer. At activity 38200, product information can be provided to a service provider, patient, and/or customer, such as via an information device (e.g., a mobile information device). At activity 38300, a test, such as a healthcare test (e.g., a vision test), can be provided and/or administered to a customer and/or patient, such as via a mobile information device. At activity 38400, test results can be provided from a mobile information device and/or reviewed by a service provider, potentially on a mobile information device. At activity 38500, a service provider can enter and/or provide recommendations to a customer and/or patient, such as via a mobile information device. At activity 38600, a customer and/or patient can communicate a purchase decision, such as via a mobile information device. At activity 38700, a service provider's recommendation(s) and/or one or more purchase decisions of one or more customers and/or patients can be communicated to an intermediary. At activity 38800, one or more service provider's recommendation(s) and/or one or more purchase decisions of one or more customers and/or patients can be reported to one or more product providers. At activity 38900, an intermediary can be compensated by one or more product providers for reporting information from one or more service provider's recommendation(s) and/or one or more purchase decisions of one or more customers and/or patients.

Certain exemplary embodiments can provide a system comprising:
a phoropter/refractor;
a near point rod attached to said phoropter/refractor; and/or
an information device directly mechanically coupled to said near point rod, said information device comprising a first hardware-containing circuit comprising an electrically conductive pathway and operatively adapted to facilitate a test, administered by an eyecare service provider, of at least one eye of a patient who views a display of said information device.

Certain exemplary embodiments can provide a method, circuit, and/or machine-readable medium storing machine-implementable instructions for activities that can comprise:
via a predetermined computer, automatically transforming information received by a healthcare information intermediary into a report having a predetermined structure and targeted to a predetermined eyecare product provider, the received information comprising:
   information, received from the predetermined eyecare product provider, regarding a particular eyecare product; and/or
   information, sent via a predetermined mobile information device associated with a predetermined eyecare service provider, regarding:
      a recommendation of the particular eyecare product, the recommendation provided by the eyecare service provider to a patient, the recommendation based on a test, administered by the eyecare service provider, of at least one eye of the patient, the recommendation communicated to the patient via the mobile information device, the eyecare service provider not compensated by the healthcare information intermediary or the eyecare product provider for recommending the particular eyecare product; and/or
      a purchase decision of the patient regarding the particular eyecare product, the purchase decision communicated from the patient and/or the eye care service provider via the mobile information device.

Certain exemplary embodiments can provide a method, circuit, and/or machine-readable medium storing machine-implementable instructions for activities that can comprise:
receiving, by a healthcare information intermediary from an eyecare product provider:
   information regarding an eyecare product; and/or
   compensation for providing, to the eyecare product provider, information regarding a recommendation of the eyecare product, the recommendation provided by an eyecare service provider to a patient, the recommendation based on a test of at least one eye of the patient, the test administered by the eyecare service provider, the recommendation communicated to the healthcare information intermediary, the recommendation communicated via a mobile information device, information regarding a purchase decision of the patient and/or the recommendation communicated to the healthcare information intermediary;
providing the mobile information device to the eyecare service supplier; and/or, wherein:
   the eyecare service provider is not compensated by the healthcare information intermediary or the eyecare product provider for recommending the eyecare product;
   the test is provided via the mobile information device;
   the test is related to a vision disease, abnormality, or refractive error;
   the test is a near acuity vision test;
   the test is a near point convergence test;
   the test is a test for retina disease;
   the test is a test for optic nerve disease;
   the test is a macular degeneration test;
   the test presents an Amsler Grid;
   the test is a cataract detection test;
   the test is a color vision deficiency test;
   the test is an opacity test;
   the test is a test for refractive error;
   the test is a light adaptation test;
   the test is a dark adaptation test;
   the test is a glare test;
   the test is a test for higher order aberration;
   the test is a test for lower order aberration;
   the test is a test for binocularity
   the test is a test for stereopsis
   the information regarding the eyecare product is provided to the patient via the mobile information device;
   the recommendation is based on personal information of the patient, the personal information entered by the patient and/or the eye care service provider via the mobile information device;
   the recommendation comprises a prescription;
   the recommendation comprises at least one: vision corrective lens power, vision corrective lens tint, lens coating, vision corrective lens material, eyeglasses, eyeglasses frame, contact lens, vision training, surgical procedure, and/or eye medication;
   the recommendation is communicated to the patient via the mobile information device;
   the information regarding the recommendation is communicated automatically to the healthcare information intermediary;
   the information regarding the recommendation lacks information sufficient to uniquely identify the patient;
   the information regarding the purchase decision is communicated to the healthcare information intermediary via the mobile information device;
   the information regarding the purchase decision is communicated automatically from the patient and/or eye care service provider to the healthcare information intermediary; and/or
   the information regarding the purchase decision lacks information sufficient to uniquely identify the patient.

Certain exemplary embodiments can provide a method, circuit, and/or machine-readable medium storing machine-implementable instructions for activities that can comprise:
providing, from an eyecare product provider to a healthcare information intermediary:
   information regarding an eyecare product; and
   compensation for providing to the eyecare product provider:
      information regarding a recommendation of the eyecare product, the recommendation provided by an eyecare service provider to a patient, the recommendation based on a test of at least one eye of the patient, the test administered by the eyecare service provider, the recommendation communicated to the healthcare information intermediary, the recommendation communicated via the mobile information device, information regarding a purchase decision of the patient and/or the recommendation communicated to the healthcare information intermediary.

### DEFINITIONS

When the following phrases are used substantively herein, the accompanying definitions apply. These phrases and definitions are presented without prejudice, and, consistent with the application, the right to redefine these phrases via amendment during the prosecution of this application or any application claiming priority hereto is reserved. For the purpose of interpreting a claim of any patent that claims priority hereto, each definition in that patent functions as a clear and unambiguous disavowal of the subject matter outside of that definition.
a—at least one.
abnormality—with respect to the typical and/or usual, a deviation, irregularity, deformity, and/or physical malformation, of a feature and/or event.
accommodation—The automatic adjustment in the focal length of the lens of the eye to permit retinal focus of images of objects at varying distances.
activity—an action, act, step, and/or process or portion thereof
acuity—clarity or clearness, especially of vision.
adapted to—suitable, fit, and/or capable of performing a specified function.
administer—to manage, supervise, oversee, run, and/or implement.
Amsler grid—a grid of horizontal and vertical lines used to monitor a person's central visual field. It is a diagnostic tool that aids in the detection of visual disturbances caused by changes in the retina, particularly the macula (e.g. macular degeneration, Epiretinal membrane), as well as the optic nerve and the visual pathway to the brain. In the test, the person looks with each eye separately at the little dot in the center of the grid. Patients might notice that they don't see the lines as straight, or lines are missing.
an—at least one.
and—in conjunction with.
and/or—either in conjunction with or in alternative to.
apparatus—an appliance or device for a particular purpose
associated—related to and/or accompanying.
at—in, on, and/or near.
at least—not less than, and possibly more than.
automatic—performed via an information device in a manner essentially independent of influence and/or control by a user. For example, an automatic light switch can turn on upon “seeing” a person in its “view”, without the person manually operating the light switch.
based—being derived from, conditional upon, and/or dependent upon.
binocular—relating to, used by, or involving both eyes at the same time.
Boolean logic—a complete system for logical operations.
by—with the use of.
can—is capable of, in at least some embodiments.
cataract—an opacity of the lens and/or capsule of the eye, causing impairment of vision or blindness.
circuit—a physical system comprising, depending on context: an electrically conductive pathway, an information transmission mechanism, and/or a communications connection, the pathway, mechanism, and/or connection established via a switching device (such as a switch, relay, transistor, and/or logic gate, etc.); and/or an electrically conductive pathway, an information transmission mechanism, and/or a communications connection, the pathway, mechanism, and/or connection established across two or more switching devices comprised by a network and between corresponding end systems connected to, but not comprised by the network.
color vision deficiency—a genetic inability to distinguish differences in hue.
communicate—to transmit and/or exchange data and/or information.
compensate—to provide and/or give payment, credit, reimbursement, and/or reparation in exchange for one or more products, services, expenditures, and/or losses.
compensation—the act of compensating, the state of being compensated, and/or something, such as money, given and/or received as payment, credit, reimbursement, and/or reparation in exchange for one or more products, services, expenditures, and/or losses.
comprising—including but not limited to.
containing—including but not limited to.
convergence—the coordinated turning of the eyes inward to focus on an object at close range.
cornea—the transparent convex anterior portion of the outer fibrous coat of the eyeball that covers the iris and the pupil and is continuous with the sclera. The cornea can have an opacity.
corrective—tending, adapted to, and/or intended to correct.
cycloplegia—paralysis of the ciliary muscle of the eye, resulting in a loss of accommodation (the process by which the eye increases optical power to maintain a clear image (focus) on an object as it draws near the eye).
cycloplegic refraction—a type of static refraction, measured after lens accommodation is paralyzed by administration of cycloplegic eyedrops.
data—distinct pieces of information, usually formatted in a special or predetermined way and/or organized to express concepts, and/or represented in a form suitable for processing by an information device.
data structure—an organization of a collection of data that allows the data to be manipulated effectively and/or a logical relationship among data elements that is designed to support specific data manipulation functions. A data structure can comprise meta data to describe the properties of the data structure. Examples of data structures can include: array, dictionary, graph, hash, heap, linked list, matrix, object, queue, ring, stack, tree, and/or vector.
decision—a passing of judgment on an issue under consideration.
device—a machine, manufacture, and/or collection thereof that is typically adapted to a particular purpose.
digital—non-analog and/or discrete.
disease—a pathological condition of a part, organ, and/or system of an organism resulting from various causes, such as infection, genetic defect, and/or environmental stress, and characterized by an identifiable group of signs and/or symptoms.
enter—to write, type, record, and/or put into.
eye—an organ of vision and/or light sensitivity; and/or either of a pair of hollow structures located in bony sockets of the skull, functioning together or independently, each having a lens capable of focusing incident light on an internal photosensitive retina from which nerve impulses are sent to the brain.
eyecare—of or relating to the care and treatment of the eyes and related structures, vision, visual systems, and/or vision information processing, typically in humans only, but potentially in any primate, mammal, animal, pet, and/or domesticated creature, and typically provided via the services of ophthalmologists, optometrists, physican's assistants, nurses, therapists, counselors, hygenists, opticians, and/or technicians, etc., and/or via any device provided, utilized, and/or administered by such service providers.
eyeglasses—spectacles and/or an optical instrument comprising a frame that holds a pair of lenses that are typically used for correcting defective vision.
field—an area visible from a point of view of an eye.
for—with a purpose of.
frame—a structure, often formed from metal, plastic, tortoiseshell, wood, and/or leather, etc., that is adapted for enclosing and/or supporting ophthalmic lenses but usually considered without the lenses.
from—used to indicate a source.
haptic—involving the human sense of kinesthetic movement and/or the human sense of touch. Among the many potential haptic experiences are numerous sensations, body-positional differences in sensations, and time-based changes in sensations that are perceived at least partially in non-visual, non-audible, and non-olfactory manners, including the experiences of tactile touch (being touched), active touch, grasping, pressure, friction, traction, slip, stretch, force, torque, impact, puncture, vibration, motion, acceleration, jerk, pulse, orientation, limb position, gravity, texture, gap, recess, viscosity, pain, itch, moisture, temperature, thermal conductivity, and thermal capacity.
having—possessing, characterized by, comprising, and/or including, but not limited to.
healthcare—of and/or relating to the prevention, treatment, and/or management of illness and/or the preservation of mental and/or physical well-being through the services offered by the medical and/or allied health professions, including services offered by one or more physicians, dentists, optometrists, ophthalmologists, veterinarians, physician's assistants, nurses, nutritionists, therapists, counselors, hygienists, pharmacists, opticians, healers, and/or technicians, etc. Examples of healthcare include medical care, dental care, vision care, psychological care, physical therapy, speech therapy, occupational therapy, mental care, etc.
healthcare service provider—one that furnishes and/or supplies one or more healthcare services relating to the prevention, treatment, and/or management of illness and/or the preservation of mental and/or physical well-being through the services offered by the medical and/or allied health professions, such as a physician, dentist, optometrist, ophthalmologist, veterinarian, physician's assistant, nurse, nutritionist, therapist, counselor, hygienist, pharmacist, optician, healer, and/or technician, etc.
human-machine interface—hardware and/or software adapted to render information to a user and/or receive information from the user; and/or a user interface.
identify—to specify, recognize, detect, and/or establish the identity, origin, nature, and/or definitive characteristics of.
including—including but not limited to.
information—facts, terms, concepts, phrases, expressions, commands, numbers, characters, and/or symbols, etc., that are related to a subject. Sometimes used synonymously with data, and sometimes used to describe data that has been organized, transformed, and/or processed to express concepts, such as according to semantic rules. It is generally possible to automate certain activities involving the management, organization, storage, transformation, communication, and/or presentation of information.
information device—any device capable of processing data and/or information, such as any general purpose and/or special purpose computer, such as a personal computer, workstation, server, minicomputer, mainframe, supercomputer, computer terminal, laptop, wearable computer, and/or Personal Digital Assistant (PDA), mobile terminal, Bluetooth device, communicator, “smart” phone (such as an iPhone-like and/or Treo-like device), messaging service (e.g., Blackberry) receiver, pager, facsimile, cellular telephone, a traditional telephone, telephonic device, a programmed microprocessor or microcontroller and/or peripheral integrated circuit elements, an ASIC or other integrated circuit, a hardware electronic logic circuit such as a discrete element circuit, and/or a programmable logic device such as a PLD, PLA, FPGA, or PAL, or the like, etc. In general any device on which resides a finite state machine capable of implementing at least a portion of a method, structure, and/or or graphical user interface described herein may be used as an information device. An information device can comprise components such as one or more network interfaces, one or more processors, one or more memories containing instructions, and/or one or more input/output (I/O) devices, one or more user interfaces coupled to an I/O device, etc.
input/output (I/O) device—any device adapted to provide input to, and/or receive output from, an information device. Examples can include an audio, visual, haptic, olfactory, and/or taste-oriented device, including, for example, a monitor, display, projector, overhead display, keyboard, keypad, mouse, trackball, joystick, gamepad, wheel, touchpad, touch panel, pointing device, microphone, speaker, video camera, camera, scanner, printer, switch, relay, haptic device, vibrator, tactile simulator, and/or tactile pad, potentially including a port to which an I/O device can be attached or connected.
instructions—directions, which can be implemented as hardware, firmware, and/or software, the directions adapted to perform a particular operation and/or function via creation and/or maintenance of a predetermined physical circuit.
intermediary—an actor logically located between actors that function as endpoints in an overall process, yet not including an endpoint actor.
into—to a condition, state, or form of
irregularity—a state or quality of being irregular and/or something irregular, such as a bump in a smooth surface.
lack—a particular deficiency or absence.
lens—a piece of transparent substance, often glass and/or plastic, having two opposite surfaces either both curved or one curved and one plane, used in an optical device for changing the convergence and/or focal point of light rays; and/or an optical device with approximate axial symmetry that transmits light, refracts light, and is adapted to cause the light to concentrate and/or diverge.
logic gate—a physical device adapted to perform a logical operation on one or more logic inputs and to produce a single logic output, which is manifested physically. Because the output is also a logic-level value, an output of one logic gate can connect to the input of one or more other logic gates, and via such combinations, complex operations can be performed. The logic normally performed is Boolean logic and is most commonly found in digital circuits. The most common implementations of logic gates are based on electronics using resistors, transistors, and/or diodes, and such implementations often appear in large arrays in the form of integrated circuits (a.k.a., IC's, microcircuits, microchips, silicon chips, and/or chips). It is possible, however, to create logic gates that operate based on vacuum tubes, electromagnetics (e.g., relays), mechanics (e.g., gears), fluidics, optics, chemical reactions, and/or DNA, including on a molecular scale. Each electronically-implemented logic gate typically has two inputs and one output, each having a logic level or state typically physically represented by a voltage. At any given moment, every terminal is in one of the two binary logic states (“false” (a.k.a., “low” or “0”) or “true” (a.k.a., “high” or “1”), represented by different voltage levels, yet the logic state of a terminal can, and generally does, change often, as the circuit processes data. Thus, each electronic logic gate typically requires power so that it can source and/or sink currents to achieve the correct output voltage. Typically, machine-implementable instructions are ultimately encoded into binary values of “0″s and/or “1”s and, are typically written into and/or onto a memory device, such as a “register”, which records the binary value as a change in a physical property of the memory device, such as a change in voltage, current, charge, phase, pressure, weight, height, tension, level, gap, position, velocity, momentum, force, temperature, polarity, magnetic field, magnetic force, magnetic orientation, reflectivity, molecular linkage, molecular weight, etc. An exemplary register might store a value of “01101100”, which encodes a total of 8 “bits” (one byte), where each value of either “0” or “1” is called a “bit” (and 8 bits are collectively called a “byte”). Note that because a binary bit can only have one of two different values (either “0” or “1”), any physical medium capable of switching between two saturated states can be used to represent a bit. Therefore, any physical system capable of representing binary bits is able to represent numerical quantities, and potentially can manipulate those numbers via particular encoded machine-implementable instructions. This is one of the basic concepts underlying digital computing. At the register and/or gate level, a computer does not treat these “0”s and “1”s as numbers per se, but typically as voltage levels (in the case of an electronically-implemented computer), for example, a high voltage of approximately +3 volts might represent a “1” or “logical true” and a low voltage of approximately 0 volts might represent a “0” or “logical false” (or vice versa, depending on how the circuitry is designed). These high and low voltages (or other physical properties, depending on the nature of the implementation) are typically fed into a series of logic gates, which in turn, through the correct logic design, produce the physical and logical results specified by the particular encoded machine-implementable instructions. For example, if the encoding request a calculation, the logic gates might add the first two bits of the encoding together, produce a result “1” (“0”+“1”=“1”), and then write this result into another register for subsequent retrieval and reading. Or, if the encoding is a request for some kind of service, the logic gates might in turn access or write into some other registers which would in turn trigger other logic gates to initiate the requested service.
logical—a conceptual representation.
machine-implementable instructions—directions adapted to cause a machine, such as an information device, to perform one or more particular activities, operations, and/or functions via forming a particular physical circuit. The directions, which can sometimes form an entity called a “processor”, “kernel”, “operating system”, “program”, “application”, “utility”, “subroutine”, “script”, “macro”, “file”, “project”, “module”, “library”, “class”, and/or “object”, etc., can be embodied and/or encoded as machine code, source code, object code, compiled code, assembled code, interpretable code, and/or executable code, etc., in hardware, firmware, and/or software.
machine-readable medium—a physical structure from which a machine, such as an information device, computer, microprocessor, and/or controller, etc., can store and/or obtain one or more machine-implementable instructions, data, and/or information. Examples include a memory device, punch card, player-piano scroll, etc.
macular degeneration—a medical condition predominantly found in elderly adults in which the center of the inner lining of the eye, known as the macula area of the retina, suffers thinning, atrophy, and in some cases, bleeding. This can result in loss of central vision, which entails inability to see fine details, to read, and/or to recognize faces.
material—a substance and/or composition.
may—is allowed and/or permitted to, in at least some embodiments.
medication—a substance adapted to relieve at least one symptom of and/or cure a medical condition.
memory device—an apparatus capable of storing, sometimes permanently, machine-implementable instructions, data, and/or information, in analog and/or digital format. Examples include at least one non-volatile memory, volatile memory, register, relay, switch, Random Access Memory, RAM, Read Only Memory, ROM, flash memory, magnetic media, hard disk, floppy disk, magnetic tape, optical media, optical disk, compact disk, CD, digital versatile disk, DVD, and/or raid array, etc. The memory device can be coupled to a processor and/or can store and provide instructions adapted to be executed by processor, such as according to an embodiment disclosed herein.
method—one or more acts that are performed upon subject matter to be transformed to a different state or thing and/or are tied to a particular apparatus, said one or more acts not a fundamental principal and not pre-empting all uses of a fundamental principal.
mobile—adapted to communicatively couple to a device and/or network via a wireless connection, such as a radio, cordless, cellular, optical, laser, visible light, infra-red, and/or acoustic connection, such as according to a protocol such as 3G, 4G, GSM, CDMA, UMTS, spread-spectrum, OFDM, Wi-Fi, WiMax, BlueTooth, Airport, IEEE 802.11, IEEE 802.11a, IEEE 802.11b, IEEE 802.11g, 802.11n, and/or X-10.
monocular—relating to, used by, or involving one eye.
near—within approximately 4 feet.
near point—the closest point at which an object can be brought into focus by the eye.
network—a communicatively coupled plurality of nodes, communication devices, and/or information devices. Via a network, such nodes and/or devices can be linked, such as via various wireline and/or wireless media, such as cables, telephone lines, power lines, optical fibers, radio waves, and/or light beams, etc., to share resources (such as printers and/or memory devices), exchange files, and/or allow electronic communications therebetween. A network can be and/or can utilize any of a wide variety of sub-networks and/or protocols, such as a circuit switched, public-switched, packet switched, connection-less, wireless, virtual, radio, data, telephone, twisted pair, POTS, non-POTS, DSL, cellular, telecommunications, video distribution, cable, radio, terrestrial, microwave, broadcast, satellite, broadband, corporate, global, national, regional, wide area, backbone, packet-switched TCP/IP, IEEE 802.03, Ethernet, Fast Ethernet, Token Ring, local area, wide area, IP, public Internet, intranet, private, ATM, Ultra Wide Band (UWB), Wi-Fi, WiMax, BlueTooth, Airport, IEEE 802.11, IEEE 802.11a, IEEE 802.11b, IEEE 802.11g, 802.11n, X-10, electrical power, 3G, 4G, multi-domain, and/or multi-zone sub-network and/or protocol, one or more Internet service providers, one or more network interfaces, and/or one or more information devices, such as a switch, router, and/or gateway not directly connected to a local area network, etc., and/or any equivalents thereof.
network interface—any physical and/or logical device, system, and/or process capable of coupling an information device to a network. Exemplary network interfaces comprise a telephone, cellular phone, cellular modem, telephone data modem, fax modem, wireless transceiver, communications port, ethernet card, cable modem, digital subscriber line interface, bridge, hub, router, or other similar device, software to manage such a device, and/or software to provide a function of such a device.
not—a negation of something.
one—being or amounting to a single unit, individual, and/or entire thing, item, and/or object.
opacity—the degree to which something reduces the passage of light.
operatively—in a manner able to function and/or to work.
or—used to indicate alternatives, typically appearing only before the last item in a group of alternative items.
packet—a generic term for a bundle of data organized in a specific way for transmission, such as within and/or across a network, such as a digital packet-switching network, and comprising the data to be transmitted and certain control information, such as a destination address.
patient—one who is scheduled to receive, has been admitted to receive, or has received, health care, and/or a human or other type of animal under supervision for health care purposes.
perceptible—capable of being perceived by the human senses.
personal—of or relating to a particular person.
physical—tangible, real, and/or actual.
physically—existing, happening, occurring, acting, and/or operating in a manner that is tangible, real, and/or actual.
plurality—the state of being plural and/or more than one.
point—a defined physical and/or logical location in at least a two-dimensional system and/or an element in a geometrically described set and/or a measurement.
power—a measure of an ability of a vision system, eye, lens, and/or lens-assisted eye, to refract, magnify, separate, converge, and/or diverge; and/or a general term that may refer to any power such as effective, equivalent, dioptric, focal, refractive, surface, and/or vergence power.
predetermined—determined, decided, obtained, calculated, and/or established in advance.
predetermined—established in advance.
prescription—a written order, especially by a health care provider, for the preparation and/or administration of a health care product, service, treatment, and/or procedure.
probability—a quantitative representation of a likelihood of an occurrence.
processor—a machine that utilizes hardware, firmware, and/or software and is physically adaptable to perform, via Boolean logic operating on a plurality of logic gates that form particular physical circuits, a specific task defined by a set of machine-implementable instructions. A processor can utilize mechanical, pneumatic, hydraulic, electrical, magnetic, optical, informational, chemical, and/or biological principles, mechanisms, adaptations, signals, inputs, and/or outputs to perform the task(s). In certain embodiments, a processor can act upon information by manipulating, analyzing, modifying, and/or converting it, transmitting the information for use by machine-implementable instructions and/or an information device, and/or routing the information to an output device. A processor can function as a central processing unit, local controller, remote controller, parallel controller, and/or distributed controller, etc. Unless stated otherwise, the processor can be a general-purpose device, such as a microcontroller and/or a microprocessor, such the Pentium family of microprocessor manufactured by the Intel Corporation of Santa Clara, Calif. In certain embodiments, the processor can be dedicated purpose device, such as an Application Specific Integrated Circuit (ASIC) or a Field Programmable Gate Array (FPGA) that has been designed to implement in its hardware and/or firmware at least a part of an embodiment disclosed herein. A processor can reside on and use the capabilities of a controller.
product—something produced by human effort.
provide—to furnish, supply, give, convey, send, and/or make available.
provider—an entity that furnishes and/or supplies one or more products and/or services.
purchase—to buy and/or to obtain one or more possession rights in exchange for money or an equivalent of money.
receiving—obtaining, taking, and/or acquiring.
recommend—to suggest, praise, commend, and/or endorse.
regarding—pertaining to.
related—connected to and/or associated with.
render—to, e.g., physically, chemically, biologically, electronically, electrically, magnetically, optically, acoustically, fluidically, and/or mechanically, etc., transform information into a form perceptible to a human as, for example, data, commands, text, graphics, audio, video, animation, and/or hyperlinks, etc., such as via a visual, audio, and/or haptic, etc., means and/or depiction, such as via a display, monitor, electric paper, ocular implant, cochlear implant, speaker, vibrator, shaker, force-feedback device, stylus, joystick, steering wheel, glove, blower, heater, cooler, pin array, tactile touchscreen, etc.
repeatedly—again and again; repetitively.
report—a presentation of information in a predetermined format.
scar—a mark and/or lingering sign left on a tissue after a surface injury, damage, and/or wound has healed.
send—to convey, dispatch, communicate, and/or transmit.
server—an information device and/or a process running thereon, that is adapted to be communicatively coupled to a network and that is adapted to provide at least one service for at least one client, i.e., for at least one other information device communicatively coupled to the network and/or for at least one process running on another information device communicatively coupled to the network. One example is a file server, which has a local drive and services requests from remote clients to read, write, and/or manage files on that drive. Another example is an e-mail server, which provides at least one program that accepts, temporarily stores, relays, and/or delivers e-mail messages. Still another example is a database server, which processes database queries. Yet another example is a device server, which provides networked and/or programmable: access to, and/or monitoring, management, and/or control of, shared physical resources and/or devices, such as information devices, printers, modems, scanners, projectors, displays, lights, cameras, security equipment, proximity readers, card readers, kiosks, POS/retail equipment, phone systems, residential equipment, HVAC equipment, medical equipment, laboratory equipment, industrial equipment, machine tools, pumps, fans, motor drives, scales, programmable logic controllers, sensors, data collectors, actuators, alarms, annunciators, and/or input/output devices, etc.
service—(n) useful work that does not necessarily produce a tangible commodity, work done for the benefit of others, and/or a performance of work and/or duties; (v) to produce a tangible commodity, to provide work for the benefit of others, and/or to perform work and/or duties.
set—a related plurality.
signal—(v) to communicate; (n) one or more automatically detectable variations in a physical variable, such as a pneumatic, hydraulic, acoustic, fluidic, mechanical, electrical, magnetic, optical, chemical, and/or biological variable, such as power, energy, pressure, flowrate, viscosity, density, torque, impact, force, frequency, phase, voltage, current, resistance, magnetomotive force, magnetic field intensity, magnetic field flux, magnetic flux density, reluctance, permeability, index of refraction, optical wavelength, polarization, reflectance, transmittance, phase shift, concentration, and/or temperature, etc., that can encode information, such as machine-implementable instructions for activities and/or one or more letters, words, characters, symbols, signal flags, visual displays, and/or special sounds, etc., having prearranged meaning. Depending on the context, a signal and/or the information encoded therein can be synchronous, asynchronous, hard real-time, soft real-time, non-real time, continuously generated, continuously varying, analog, discretely generated, discretely varying, quantized, digital, broadcast, multicast, unicast, transmitted, conveyed, received, continuously measured, discretely measured, processed, encoded, encrypted, multiplexed, modulated, spread, de-spread, demodulated, detected, de-multiplexed, decrypted, and/or decoded, etc.
special purpose computer—a computer and/or information device comprising a processor device having a plurality of logic gates, whereby at least a portion of those logic gates, via implementation of specific machine-implementable instructions by the processor, experience a change in at least one physical and measurable property, such as a voltage, current, charge, phase, pressure, weight, height, tension, level, gap, position, velocity, momentum, force, temperature, polarity, magnetic field, magnetic force, magnetic orientation, reflectivity, molecular linkage, molecular weight, etc., thereby directly tying the specific machine-implementable instructions to the logic gate's specific configuration and property(ies). In the context of an electronic computer, each such change in the logic gates creates a specific electrical circuit, thereby directly tying the specific machine-implementable instructions to that specific electrical circuit.
special purpose processor—a processor device, having a plurality of logic gates, whereby at least a portion of those logic gates, via implementation of specific machine-implementable instructions by the processor, experience a change in at least one physical and measurable property, such as a voltage, current, charge, phase, pressure, weight, height, tension, level, gap, position, velocity, momentum, force, temperature, polarity, magnetic field, magnetic force, magnetic orientation, reflectivity, molecular linkage, molecular weight, etc., thereby directly tying the specific machine-implementable instructions to the logic gate's specific configuration and property(ies). In the context of an electronic computer, each such change in the logic gates creates a specific electrical circuit, thereby directly tying the specific machine-implementable instructions to that specific electrical circuit.
store—to place, hold, and/or retain data, typically in a memory.
structure—a hierarchy and/or placement of objects in a document; and/or a manner in which objects and/or components are organized and/or form a whole.
substantially—to a great extent and/or degree.
sufficient—a degree and/or amount necessary to achieve a predetermined result.
supplier—a provider and/or an entity that provides, furnishes, and/or supplies one or more products and/or services.
switch—(v) to: form, open, and/or close one or more circuits; form, complete, and/or break an electrical and/or informational path; select a path and/or circuit from a plurality of available paths and/or circuits; and/or establish a connection between disparate transmission path segments in a network (or between networks); (n) a physical device, such as a mechanical, electrical, and/or electronic device, that is adapted to switch.
system—a collection of mechanisms, devices, machines, articles of manufacture, processes, data, and/or instructions, the collection designed to perform one or more specific functions.
test—to evaluate and/or determine.
tint—a shade or gradation of a color; a slight coloration; and/or a trace, tinge, and/or barely detectable amount and/or degree of color.
to—a preposition adapted for use for expressing purpose.
transform—to change in measurable: form, appearance, nature, and/or character.
uniquely—unambiguously, clearly, and/or with a very low margin for error.
user interface—any device for rendering information to a user and/or requesting information from the user. A user interface includes at least one of textual, graphical, audio, video, animation, and/or haptic elements. A textual element can be provided, for example, by a printer, monitor, display, projector, etc. A graphical element can be provided, for example, via a monitor, display, projector, and/or visual indication device, such as a light, flag, beacon, etc. An audio element can be provided, for example, via a speaker, microphone, and/or other sound generating and/or receiving device. A video element or animation element can be provided, for example, via a monitor, display, projector, and/or other visual device. A haptic element can be provided, for example, via a very low frequency speaker, vibrator, tactile stimulator, tactile pad, simulator, keyboard, keypad, mouse, trackball, joystick, gamepad, wheel, touchpad, touch panel, pointing device, and/or other haptic device, etc. A user interface can include one or more textual elements such as, for example, one or more letters, number, symbols, etc. A user interface can include one or more graphical elements such as, for example, an image, photograph, drawing, icon, window, title bar, panel, sheet, tab, drawer, matrix, table, form, calendar, outline view, frame, dialog box, static text, text box, list, pick list, pop-up list, pull-down list, menu, tool bar, dock, check box, radio button, hyperlink, browser, button, control, palette, preview panel, color wheel, dial, slider, scroll bar, cursor, status bar, stepper, and/or progress indicator, etc. A textual and/or graphical element can be used for selecting, programming, adjusting, changing, specifying, etc. an appearance, background color, background style, border style, border thickness, foreground color, font, font style, font size, alignment, line spacing, indent, maximum data length, validation, query, cursor type, pointer type, autosizing, position, and/or dimension, etc. A user interface can include one or more audio elements such as, for example, a volume control, pitch control, speed control, voice selector, and/or one or more elements for controlling audio play, speed, pause, fast forward, reverse, etc. A user interface can include one or more video elements such as, for example, elements controlling video play, speed, pause, fast forward, reverse, zoom-in, zoom-out, rotate, and/or tilt, etc. A user interface can include one or more animation elements such as, for example, elements controlling animation play, pause, fast forward, reverse, zoom-in, zoom-out, rotate, tilt, color, intensity, speed, frequency, appearance, etc. A user interface can include one or more haptic elements such as, for example, elements utilizing tactile stimulus, force, pressure, vibration, motion, displacement, temperature, etc.
via—by way of, with, and/or utilizing.
vision—eyesight and/or the faculty of sight.
visual field—a spatial array of visual sensations available to observation in introspectionist psychological experiments and/or a brain-processed output based on inputs provided to the visual system. The normal human visual field extends to approximately 60 degrees nasally (toward the nose, or inward) in each eye, to approximately 100 degrees temporally (away from the nose, or outwards), and approximately 60 degrees above and approximately 75 below the horizontal meridian.
visual field test—a test used to determine whether the visual field is affected by diseases that cause local scotoma, a more extensive loss of vision, and/or a reduction in sensitivity (threshold).
wherein—in regard to which; and; and/or in addition to.
with—accompanied by.

### NOTE

Various substantially and specifically practical and useful exemplary embodiments of the claimed subject matter are described herein, textually and/or graphically, including the best mode, if any, known to the inventor(s), for implementing the claimed subject matter by persons having ordinary skill in the art. Any of numerous possible variations (e.g., modifications, augmentations, embellishments, refinements, and/or enhancements, etc.), details (e.g., species, aspects, nuances, and/or elaborations, etc.), and/or equivalents (e.g., substitutions, replacements, combinations, and/or alternatives, etc.) of one or more embodiments described herein might become apparent upon reading this document to a person having ordinary skill in the art, relying upon his/her expertise and/or knowledge of the entirety of the art and without exercising undue experimentation. The inventor(s) expects skilled artisans to implement such variations, details, and/or equivalents as appropriate, and the inventor(s) therefore intends for the claimed subject matter to be practiced other than as specifically described herein. Accordingly, as permitted by law, the claimed subject matter includes and covers all variations, details, and equivalents of that claimed subject matter. Moreover, as permitted by law, every combination of the herein described characteristics, functions, activities, substances, and/or structural elements, and all possible variations, details, and equivalents thereof, is encompassed by the claimed subject matter unless otherwise clearly indicated herein, clearly and specifically disclaimed, or otherwise clearly contradicted by context.

The use of any and all examples, or exemplary language (e.g., “such as”) provided herein, is intended merely to better illuminate one or more embodiments and does not pose a limitation on the scope of any claimed subject matter unless otherwise stated. No language herein should be construed as indicating any non-claimed subject matter as essential to the practice of the claimed subject matter.

Thus, regardless of the content of any portion (e.g., title, field, background, summary, description, abstract, drawing figure, etc.) of this document, unless clearly specified to the contrary, such as via explicit definition, assertion, or argument, or clearly contradicted by context, with respect to any claim, whether of this document and/or any claim of any document claiming priority hereto, and whether originally presented or otherwise:
there is no requirement for the inclusion of any particular described characteristic, function, activity, substance, or structural element, for any particular sequence of activities, for any particular combination of substances, or for any particular interrelationship of elements;
no described characteristic, function, activity, substance, or structural element is “essential”;
any two or more described substances can be mixed, combined, reacted, separated, and/or segregated;
any described characteristics, functions, activities, substances, and/or structural elements can be integrated, segregated, and/or duplicated;
any described activity can be repeated, any activity can be performed by multiple entities, and/or any activity can be performed in multiple jurisdictions; and
any described characteristic, function, activity, substance, and/or structural element can be specifically excluded, the sequence of activities can vary, and/or the interrelationship of structural elements can vary.

The use of the terms “a”, “an”, “said”, “the”, and/or similar referents in the context of describing various embodiments (especially in the context of the following claims) are to be construed to cover both the singular and the plural, unless otherwise indicated herein or clearly contradicted by context.

The terms “comprising,” “having,” “including,” and “containing” are to be construed as open-ended terms (i.e., meaning “including, but not limited to,”) unless otherwise noted.

When any number or range is described herein, unless clearly stated otherwise, that number or range is approximate. Recitation of ranges of values herein are merely intended to serve as a shorthand method of referring individually to each separate value falling within the range, unless otherwise indicated herein, and each separate value and each separate subrange defined by such separate values is incorporated into the specification as if it were individually recited herein. For example, if a range of 1 to 10 is described, that range includes all values therebetween, such as for example, 1.1, 2.5, 3.335, 5, 6.179, 8.9999, etc., and includes all subranges therebetween, such as for example, 1 to 3.65, 2.8 to 8.14, 1.93 to 9, etc.

When any phrase (i.e., one or more words) appearing in a claim is followed by a drawing element number, that drawing element number is exemplary and non-limiting on claim scope.

No claim of this document is intended to invoke paragraph six of 35 USC 112 unless the precise phrase “means for” is followed by a gerund.

Any information in any material (e.g., a United States patent, United States patent application, book, article, etc.) that has been incorporated by reference herein, is incorporated by reference herein in its entirety to its fullest enabling extent permitted by law yet only to the extent that no conflict exists between such information and the other statements and drawings set forth herein. In the event of such conflict, including a conflict that would render invalid any claim herein or seeking priority hereto, then any such conflicting information in such material is specifically not incorporated by reference herein.

Within this document, and during prosecution of any patent application related hereto, any reference to any claimed subject matter is intended to reference the precise language of the then-pending claimed subject matter at that particular point in time only.

Accordingly, every portion (e.g., title, field, background, summary, description, abstract, drawing figure, etc.) of this document, other than the claims themselves and any provided definitions of the phrases used therein, is to be regarded as illustrative in nature, and not as restrictive. The scope of subject matter protected by any claim of any patent that issues based on this document is defined and limited only by the precise language of that claim (and all legal equivalents thereof) and any provided definition of any phrase used in that claim, as informed by the context of this document.

What is claimed is:

1. A system comprising:
a phoropter/refractor;
a near point rod attached to said phoropter/refractor; and
an information device directly mechanically coupled to said near point rod, said information device comprising a first hardware-containing circuit comprising an electrically conductive pathway and operatively adapted to facilitate a test, administered by an eyecare service provider, of at least one eye of a patient who views a display of said information device.

2. A circuit comprising:
a first hardware-containing sub-circuit comprising an electrically conductive pathway and operatively adapted to transform information received by a healthcare information intermediary into a report having a predetermined structure and targeted to a predetermined eyecare product provider, the received information comprising:
information, received from the predetermined eyecare product provider, regarding a particular eyecare product; and
information, sent via a predetermined mobile information device associated with a predetermined eyecare service provider, regarding:
a recommendation of the particular eyecare product, the recommendation provided by the eyecare service provider to a patient, the recommendation based on a test, administered by the eyecare service provider, of at least one eye of the patient, the recommendation communicated to the patient via the predetermined mobile information device, the eyecare service provider not compensated by the healthcare information intermediary or the eyecare product provider for recommending the particular eyecare product; and
a purchase decision of the patient regarding the particular eyecare product, the purchase decision communicated from the patient via the predetermined mobile information device.

3. A circuit comprising:
a first hardware sub-circuit comprising an electrically conductive pathway and operatively adapted to receive, by a healthcare information intermediary from an eyecare product provider:
information regarding an eyecare product; and
compensation for providing to the eyecare product provider:
information regarding a recommendation of the eyecare product, the recommendation provided by an eyecare service provider to a patient, the recommendation based on a test of at least one eye of the patient, the test administered by the eyecare service provider, the recommendation communicated to the healthcare information intermediary, the recommendation communicated via an information device, information regarding a purchase decision of the patient communicated to the healthcare information intermediary.

4. A method comprising:
receiving, by a healthcare information intermediary from an eyecare product provider:
information regarding an eyecare product; and
compensation for providing, to the eyecare product provider, information regarding a recommendation of the eyecare product, the recommendation provided by an eyecare service provider to a patient, the recommendation based on a test of at least one eye of the patient, the test administered by the eyecare service provider, the recommendation communicated to the healthcare information intermediary, the recommendation communicated via an information device, information regarding a purchase decision of the patient communicated to the healthcare information intermediary.

5. The method of claim 4, wherein:
the information device is a mobile information device.

6. The method of claim 4, wherein:
the eyecare service provider is not compensated by the healthcare information intermediary or the eyecare product provider for recommending the eyecare product.

7. The method of claim 4, further comprising:
providing the information device to the eyecare service supplier.

8. The method of claim 4, wherein:
the test is provided via the information device.

9. The method of claim 4, wherein:
the test is related to a vision disease, abnormality, or refractive error.

10. The method of claim 4, wherein:
the test is a near acuity vision test.

11. The method of claim 4, wherein:
the test is a near point convergence test.

12. The method of claim 4, wherein:
the test is a macular degeneration test.

13. The method of claim 4, wherein:
the test presents an Amsler Grid.

14. The method of claim 4, wherein:
the test is a cataract detection test.

15. The method of claim 4, wherein:
the test is a color vision deficiency test.

16. The method of claim 4, wherein:
the test is an opacity test.

17. The method of claim 4, wherein:
the information regarding the eyecare product is provided to the patient via the information device.

18. The method of claim 4, wherein:
the recommendation is based on personal information of the patient, the personal information entered by the patient via the information device.

19. The method of claim 4, wherein:
the recommendation comprises a prescription.

20. The method of claim 4, wherein:
the recommendation comprises at least one: vision corrective lens power, vision corrective lens tint, lens coating, vision corrective lens material, eyeglasses, eyeglasses frame, contact lens, vision training, surgical procedure, and/or eye medication.

21. The method of claim 4, wherein:
the recommendation is communicated to the patient via the information device.

22. The method of claim 4, wherein:
the information regarding the recommendation is communicated automatically to the healthcare information intermediary.

23. The method of claim 4, wherein:
the information regarding the recommendation lacks information sufficient to uniquely identify the patient.

24. The method of claim 4, wherein:
the information regarding the purchase decision is communicated to the healthcare information intermediary via the information device.

25. The method of claim 4, wherein:
the information regarding the purchase decision is communicated automatically from the patient to the healthcare information intermediary.

26. The method of claim 4, wherein:
the information regarding the purchase decision lacks information sufficient to uniquely identify the patient.

27. A machine-readable medium storing machine-implementable instructions for activities comprising:
receiving, by a healthcare information intermediary from an eyecare product provider:
information regarding an eyecare product; and
compensation for providing to the eyecare product provider:
information regarding a recommendation of the eyecare product, the recommendation provided by an eyecare service provider to a patient, the recommendation based on a test of at least one eye of the patient, the test administered by the eyecare service provider, the recommendation communicated to the healthcare information intermediary, the recommendation communicated via an information device, information regarding a purchase decision of the patient communicated to the healthcare information intermediary.

28. A method comprising:
providing, from an eyecare product provider to a healthcare information intermediary:
information regarding an eyecare product; and
compensation for providing to the eyecare product provider:
information regarding a recommendation of the eyecare product, the recommendation provided by an eyecare service provider to a patient, the recommendation based on a test of at least one eye of the patient, the test administered by the eyecare service provider, the recommendation communicated to the healthcare information intermediary, the recommendation communicated via an information device, information regarding a purchase decision of the patient communicated to the healthcare information intermediary.

29. A method comprising:
receiving, by a healthcare information intermediary from an eyecare product provider:
information regarding an eyecare product; and
compensation for providing, to the eyecare product provider, information regarding a recommendation of the eyecare product, the recommendation provided by an eyecare service provider to a patient, the recommendation based on a test of at least one eye of the patient, the test administered by the eyecare service provider, the recommendation communicated to the healthcare information intermediary, the recommendation communicated via an information device, information regarding the recommendation communicated to the healthcare information intermediary.
